(19) United States
(12) Patent Application Publication
(10) Pub. No.: US 2006/0219558 A1
(43) Pub. Date: Oct. 5, 2006

(54) Improved Methods and Devices for Concentration and Fractionation of Analytes for Chemical Analysis including Matrix-Assisted Laser Desorption/Ionization (MALDI) Mass Spectrometry (MS)

(76) Inventors: Dean G. Hafeman, Knoxville, TN (US); James B. Harkins, Knoxville, TN (US); Jeremy L. Norris, Knoxville, TN (US); Sheila N. Baker, Knoxville, TN (US); Donald Loveday, Knoxville, TN (US); Daniel Kuban, Knoxville, TN (US); Richard M. Caprioli, Brentwood, TN (US); Charles E. Witkowski, Knoxville, TN (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 11/278,799

(22) Filed: Apr. 5, 2006

Related U.S. Application Data
(60) Provisional application No. 60/668,337, filed on Apr. 5, 2005.
(60) Provisional application No. 60/668,794, filed on Apr. 6, 2005.
(60) Provisional application No. 60/712,255, filed on Aug. 29, 2005.

(51) Int. Cl. C07K 1/26; G01N 27/00

(52) U.S. Cl. 204/456; 204/606

(57) ABSTRACT

A device for pre-concentration and purification of analytes from biological samples, such as human serum, to be analyzed by Matrix-Assisted Laser Desorption Ionization Mass Spectrometry (MALDI MS) and methods of use thereof are provided.

### CROSS-REFERENCE

This application claims the benefit of U.S. Provisional Application Nos. 60/668,337, filed Apr. 5, 2005 and 60/668,794, filed Apr. 6, 2005, and 60/712,255 filed on Aug. 29, 2005 and the contents which are incorporated by reference herein in their entirety.

### BACKGROUND

1. Field of Invention

The present invention relates to Mass Spectrometry (MS) and, more specifically, to pre-concentration and purification of analytes from biological samples, such as human serum, to be analyzed by Matrix-Assisted Laser Desorption Ionization Mass Spectrometry (MALDI MS).

2. Background and Significance

Devices and methods are disclosed to facilitate the concentration and capture of proteins, peptides and other analyte molecules onto a solid capture phase from the mobile phase of electrophoretic concentrator cells. Further such solid capture phases are adaptable for direct analysis in a mass spectrometer. Mass spectrometry allows multiple analytes to be monitored simultaneously, in contrast to most other analytical techniques that quantify only one, or at most, just a few different molecules at a time. Recent advances in mass spectrometry; such as lower cost instrumentation, improved ease of use, and high throughput MALDI methods; promise to revolutionize clinical research, and then as a result the entire healthcare industry. A key to realizing this tremendous potential, however, is the development of new sample preparation technologies capable of preparing complex biological samples for mass spectrographic analysis rapidly and reproducibly. Such technologies need to accommodate a wide variety of samples including solids including tissue homogenates, whole tissue slices or other solid tissue preparations, as well as liquid samples such as whole blood, plasma, serum, cerebrospinal fluid, saliva urine and the like. Serum is perhaps the most clinically important biological fluid, with hundreds of millions of samples taken by vacuum tube yearly for medical diagnoses. Blood and lymphatic fluids are rich sources of disease biomarkers because, in addition to natural blood-borne proteins & polypeptides circulating in blood and lymph fluids, body tissues release additional cellular components into the blood and lymph streams. Thus these circulating fluids contain disease biomarkers including proteins & polypeptides (PP) that are indicative of pathological conditions, such as cellular hyperplasia, necrosis, apoptosis, or shedding of antigens from neoplastic tissue. Here the term PP is used to refer to oligopeptides or proteins of broad molecular weight range including the range of from two, or more, amino acids (i.e., of approximately 200 Daltons) to high molecular weight proteins (of about 1 million Daltons, or more).

An especially promising class of disease markers in serum are the low molecular weight (LMW) PP fragments whose abundances and structures change in ways indicative of many, if not most, human diseases¹. The LMW serum proteome is made up of several classes of physiologically important polypeptides, such as cytokines, chemokines, peptide hormones, as well as proteolytic fragments of larger proteins. These proteolytically-derived peptides have been shown to correlate with pathological conditions such as cancer, diabetes and cardiovascular and infectious diseases. Analysis of the LMW serum proteome, however, requires extensive sample preparation and is notoriously difficult to analyze due to the large proportion of albumin (˜55%) that dominates the total amount of protein in blood serum. Other problems include he wide dynamic range in abundance of other LMW PP molecules, and the tremendous heterogeneity of the dominant glycoproteins. For example, the rarest proteins now measured clinically (FIG. 1) are present at concentrations more than 10 orders of magnitude lower than albumin². These rare proteins and peptides, however, are believed to represent highly sensitive and selective disease markers and potential drug targets.
¹Tirumalai, R. S., K. C. Chan, D. A. Prieto, H. J. Issaq, T. P. Conrads and T. D. Veenstra, (2003) Characterization of the Low Molecular Weight Human Serum Proteome, Molecular & Cellular Proteomics 2, 1096-1103.

²Anderson N. L. and N. G. Anderson (2002) The Human Plasma Proteome, Molecular and Cellular Proteomics 1, 845-867.

Traditionally, liquid chromatography (LC) or affinity-based methods have been used to the greatest extent to provide for a suitable separation process. Purification via LC methods involves chemically attaching linker molecules to a stationary phase (producing a functionalized stationary phase) in a LC column. Once the sample is loaded into the column, a mobile phase is flowed through the stationary phase. The fraction of the time each analyte spends bound to the stationary phase, rather than in the mobile phase, determines the relative migration rate of different analytes (as well as contaminants and interfering species) through the LC column, providing for purification of the analytes. For example, analyte molecules of interest, such as peptides and proteins, can be adsorbed onto a functionalized stationary phase while the contaminants are eluted from the column. Next, the mobile phase is adjusted so as to release the molecules of interest from the functionalized stationary phase. Often, a volatile buffer that is compatible with MALDI-MS, such as an acetonitrile/water mixture, is used as the mobile phase in this step. In this fashion, the purified molecules of interest are eluted from the LC column and collected for MALDI-MS analysis. The sample is now relatively free of salts and other contaminants that would otherwise interfere or otherwise limit the sensitivity of the analysis. There is a need therefore, for improved devices and procedures for separating, concentrating and adding reagents needed for analysis of samples during high throughput methods of analysis. Recent reviews of sample preparation techniques for mass spectrometry show that these methods remain time-consuming, cumbersome, require highly skilled labor and are difficult to automate³,⁴. As a result, the number of samples that can be analyzed within any one clinical study is extremely limited, thus substantially hindering the level of statistical significance and, therefore, clinical relevance, of these studies. Consequently, principally due to the lack of sample preparation systems the LMW serum proteome is an excellent, largely unexplored, source of biomarkers (detectable by mass spectrometery) for disease, disease treatment and gene expression analysis in humans, as well as other animals.
³Westermeier, R. and T. Naven (2002); In: Proteomics in Practice; Wiley-VCH Verlay-GbmH, Weinheim.

⁴Hamdan, M. and P. G. Righetti (2005); In: Proteomics Today; John Wiley & Sons, Hoboken, N.J.

Matrix-assisted laser desorption/ionization mass spectrometry (MS) analysis of samples deposited onto MALDI target plates is rapidly becoming a method of choice for analysis of proteins, peptides and other biological molecules. The MALDI-MS procedure is a very sensitive analytical method and is probably the MS procedure most compatible with biological salts and pH buffers. Further, its ability to generate high-mass ions at high efficiency from sub-picomole quantities of biological macromolecules makes this technique extremely useful for macromolecule analysis. Analysis of peptide analytes in crude biological samples, such as blood, plasma, or serum, however offers special problems for mass spectrometry analysis as described below.

The first problem to be overcome is that the biological samples contain high concentrations of salts (e.g. sodium, potassium, chloride, phosphate and carbonate). The anions especially are effective in suppressing the ionization of peptide samples by the usual MALDI analysis procedures. The cations also are problematic in that they generate adduct spectra that split the primary mass peaks of proteins into a multitude of additional mass peaks each having the additional mass of one cation. Also, the success of MALDI-MS analysis depends to a great extent on the ability of the analyst technician to effectively crystallize a MALDI matrix substance mixed together with the analyte prior to injection into the mass spectrometer. The MALDI matrix substance is needed to absorb the laser light that provides for atomization and ionization of the matrix together with adsorbed analyte substances within samples to be analyzed. The ionized analyte molecules then are accelerated into a mass spectrometer ion detector by a high electrical field provided by high voltages on an anode and cathode within the mass spectrometer. When even relatively small amounts of contaminants (such as salts or glycerol) are present the ability of MALDI matrices to efficiently desorb and ionize analytes, such as proteins and peptides, is dramatically reduced. Furthermore, high salt concentrations increase both the threshold laser intensity required for MALDI-MS and the intensity of salt-adducted peptide peaks (at the expense of free peptide peaks).

Secondly, in samples, such as human serum, analyte peptides are frequently present at very low copy number compared to interfering proteins (e.g. albumin, immunoglobulins and transferin). The peptides of interest often are present at just 1 micromole per liter to 1 picomole per liter (e.g. 1 microgram to 1 picogram per ml). In contrast total albumin and gamma globulins such as IgG, IgM, are present at levels ranging from 0.01 to 0.1 grams per ml, i.e. up to 1×10¹¹-fold greater in mass. Thus, the major abundance proteins heavily dominate MALDI spectra of the mixture. Minor components are rarely observed because the low intensity peaks are obscured by the major peaks. This problem is made much more difficult in biological samples, such as human serum where such low copy number molecules need to be detected in the presence of many orders of magnitude higher molar concentrations of interfering proteins (e.g. albumin, immunoglobulins and transferin) and salts (e.g. sodium, potassium, chloride, phosphate and carbonate).

Thirdly, many of the analyte peptides are hydrophobic and are bound to the major proteins found in blood, plasma, or serum. Albumin especially tends to bind hydrophobic molecules nonspecifically. Thus, removal of the unwanted proteins such as albumin also results in the loss of analyte peptides. Chemically disruptive agents, such as salts and detergents are known to assist in the dissociation of analyte peptides from albumin. These agents actively suppress the MALDI process however. For example polyethylene glycol (PEG) and Trition ionize and desorb by MALDI as efficiently as peptides and proteins. As a result these species often compete with ionization of proteins and peptides and thereby suppress the MALDI-MS signals from the latter. Thus, after the addition of chemically disruptive agents to dissociate analyte peptides from albumin, the analyst must separate the analyte peptides from both the disruptive agent's albumin and other contaminating proteins. Additionally, the separation must be performed in such a way that the minor component peptide analytes are not lost during the separation process. This separation is made especially difficult when the analytes are hydrophobic and tend to adhere to hydrophobic surfaces. Unfortunately, purification of biopolymers by LC methods frequently results in 30%, or greater, sample losses and can add fcontaminants (or sample “cross-talk” to samples. For most MALDI-MS users, this amount of sample loss is unacceptable Fourth, because the analyte peptides are present at such low levels, they must be concentrated prior to MALDI-MS analysis. Carrying out first the dissociation of peptides, the separation of components, and then the concentration, by prior art methods is tedious and requires multiples steps that are both time-consuming and labor-intensive.

### SUMMARY OF THE INVENTION

One object of the present invention therefore is to provide methods and devices to remove salts from biological samples. A second object of the invention is to to remove high abundance molecules, such as proteins, from biological samples thereby allowing reproducible and sensitive analysis of the remaining low abundance molecules. A third object of the invention is to dissociate analyte peptides from albumin and other hydrophobic proteins. A fourth object of the invention is to concentrate analyte peptides and proteins of interest for MALDI mass spectrometry analysis. A fifth object of the invention is to provide the first four objects of the invention in a convenient and effective manner, so as to provide for high sample throughput. A sixth object of the invention is to provide for handling a multiplicity of samples simultaneously, so that two- or more samples may be analyzed in parallel. Thereby, in combination with the other objects of the invention, an analyst will be able to utilize the instant invention to perform analysis of peptides and proteins in biological tissue samples in a convenient and efficient manner, thereby increasing the sensitivity of detection, increasing the sample throughput, as well as decreasing the cost of analysis. Lastly, there is a desire for analysis of the separated analyte peptides, polypeptides and proteins (analytes) to be done reproducibly and quantitatively. Thus a seventh object of the invention is to provide for reproducible and quantitative MALDI-MS analysis of peptides and proteins in biological samples.

Employing the term PP to refer to oligopeptides ranging from small size of two, or more, amino acids to large proteins of 1 million Daltons, or more, an eight object of the invention is to provide an analysis system to examine the LMW fraction of PP in human serum by mass spectrometry (MS). A ninth object of the invention is to provide a PPAS with sufficient versatility that that a wider range of PP, for example from 500 Daltons to 500,000 Daltons, or more, also can be analyzed by mass spectrometry (MS). A tenth object of the invention is to provide improvements to the PPAS to further increase the sensitivity of detection so that quantities of PP from 1 nanomole to 0.1 attomole, or less, can be detected, quantified and molecular weight measured by MS. An eleventh object of the invention is to provide for increased fractionation and separation of PP in human serum so that low abundance PP can be separated from higher-abundance PP prior to MS analysis thus providing increased sensitivity of detection of the low abundance PP.

### DESCRIPTION OF THE FIGURES

FIG. 1: The Human Plasma Proteome shows the challenge of analyzing proteins and polypeptides present in serum as they span a range in concentrations of over 10 orders in magnitude. (The Figure is adapted from reference 2.)

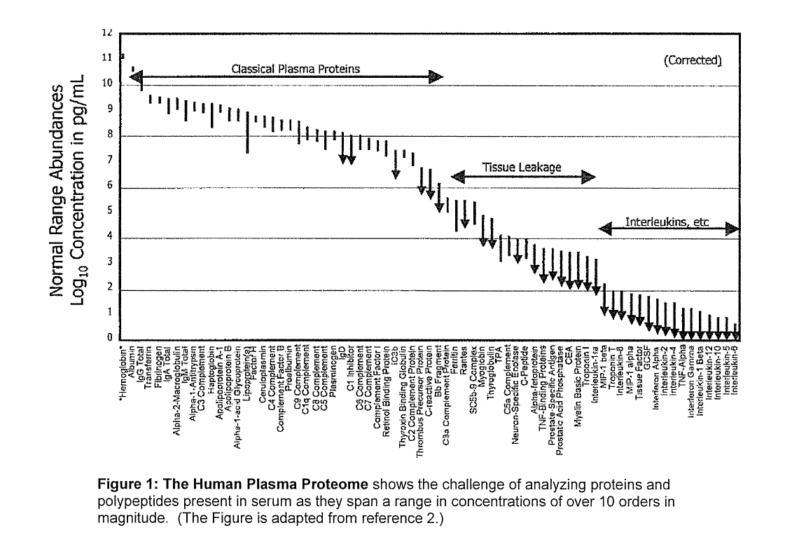

FIG. 2: Schematic cut-away drawing of a single well of an Analysis System. In a preferred embodiment of the Analysis System has a 8×12 array of 96 sample wells contained within a cartridge.

FIG. 3: Schematic drawing of an array of Sample Wells comprising the Cartridge in a preferred embodiment of the Analysis System

FIG. 4: An embodiment of Capture Slide 42 showing Apertures 50 inserted into a MALDI Slide Holder 90 having a Mechanical Guide 92.

FIG. 4a. Slide-Washing Manifold for Applying Pressure-Driven Fluid Flow Across Capture Slide

FIG. 4b. Electrophoretic Slide-Washing Apparatus for Maintaining an Electrolyte in Contact with the Capture Materials on a Capture Slide and for Applying an Electric Field in the Electrolyte Across the Capture Materials

FIG. 5: A plot of Polypeptide Standards at 1 pmol and BSA at ˜127 pmol on Steel MALDI Target Plate.

FIG. 6: A plot of Polypeptide Standards at 0.1 pmol and BSA at ˜127 pmol on Steel MALDI Target Plate.

FIG. 7: A plot of Polypeptide Standards at 0.1 pmol and BSA at ˜127 pmol concentrated with albumin depletion within a PPAS Device

FIG. 8: MALDI Mass Spectra of Serum Proteins.

FIG. 9: Binary pH Fraction using the PPAS Device

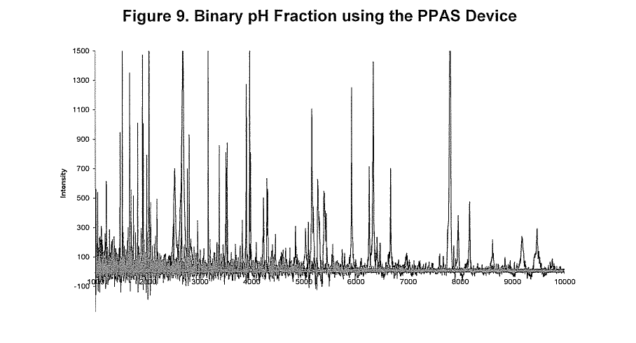

FIG. 10: Mass Spectrometry Results from the Analysis of Positively Charged LMW Proteins in Human Serum obtained with an Alpha Prototype of the Protein/Polypeptide Analysis System (PPAS) having a Single Capture Membrane as the Capture Material.

FIG. 11: Mass Spectrometry Results from the Analysis of Negatively Charged LMW Proteins in Human Serum obtained with an Alpha Prototype of the Protein/Polypeptide Analysis System (PPAS) having a Single Capture Membrane as the Capture Material.

### DETAILED DESCRIPTION OF THE PREFERRED EMBODIMENT

Incorporated in its entirety, by reference herein, is U.S. patent application Ser. No. 10/963,336, filed Oct. 12, 2004, which discloses methods and devices for use in the field of the invention. The methods and capture slides of this invention may be used in association with the apparatuses and methods disclosed therein. The methods and capture slides of this invention further may be used in association with the apparatuses disclosed in Provisional Patent Application No. 60/748,771, filed Dec. 8, 2005, which is incorporated herein in its entirety by reference.

A useful embodiment of the invention is a Peptide and Protein Analysis System (PPAS) that electrophoretically separates, concentrates and captures low abundance proteins and polypeptides present in biological samples such as serum (or from other tissues) onto a solid-phase capture slide. Following a brief rinse step, salts and other interfering molecules are washed away. Then, a MALDI matrix solution is applied to the capture slide. As is well knowin in the prior art, such matrix solutions, generally containing an organic solvent, release the proteins for incorporation into MALDI matrix crystals that precipitate on the slide surface upon drying of the solvent. Next the slide is dried completely and inserted directly into a MALDI-MS instrument for quantification of both the mass and the relative abundance of the captured proteins.

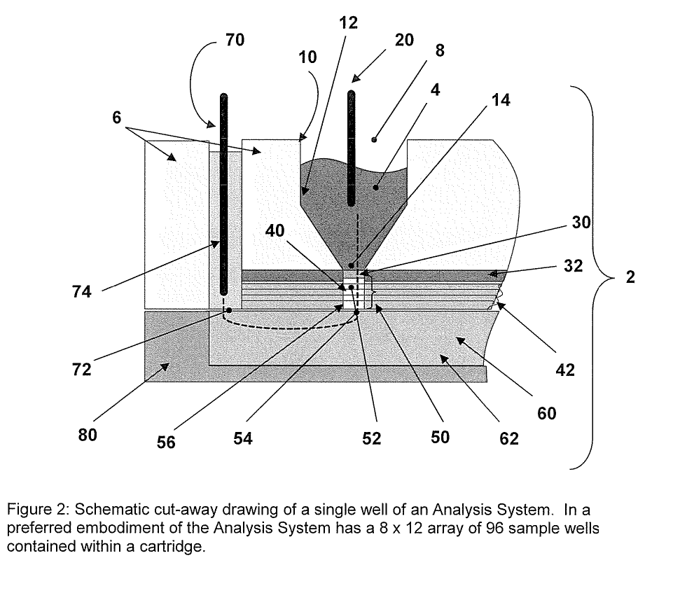

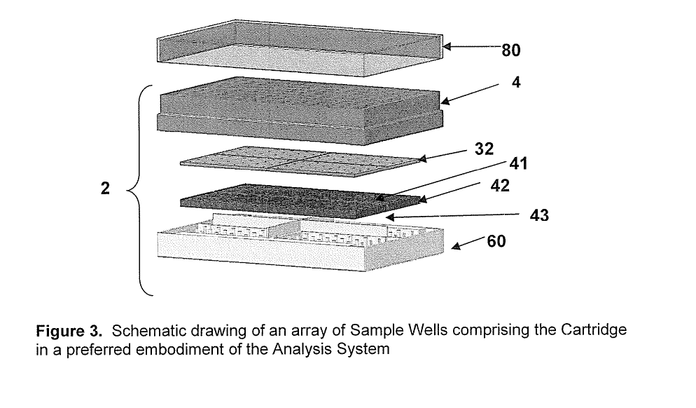

As shown in detail in FIGS. 2 and 3 the PPAS is comprised of a cartridge 2 having one, or more wells 4 for retaining fluid samples. One embodiment of the cartridge 2 includes twenty-five (25) sample wells for processing twenty-five (25) samples simultaneously. A preferred embodiment of the cartridge 2 includes ninety-six (96) sample wells in an 8×12 array for processing ninety-six (96) samples simultaneously. In the preferred embodiment the capture slides 42 and reagents needed to perform a separation and capture are predisposed as an array of sample wells 4 within cartridge 2. FIG. 3 shows an array of sample wells 4 comprising the cartridge 2. FIG. 2 shows a schematic drawing in a cut-away view of one well of a multi-well PPAS cartridge 2.

Each sample well 4 has a top opening 8, side walls 10 the bottom portion 12 which are progressively reduced in dimension from a wide top opening 8 to a narrow bottom opening 14. The top opening 8 of the sample wells 4 accepts a sample electrode 20 that makes electrical contact with electrolyte samples placed within the sample wells, as shown in FIG. 2. In a preferred embodiment of the invention each sample well is designed to hold approximately 50 μL of sample, 200 μL of electrophoresis buffer and leave 150 μL of remaining head-space (400 μL total volume). The sample electrode 20, which in a preferred embodiment is provided as an array of sample electrodes, removably fits into a top opening 8 of each sample well 4. The array of sample electrodes is designed to be reusable and cleanable by simply rinsing the assembly with DI water, or other suitable solvent, prior to each use. Optionally a more stringent cleaning may be performed either with detergents, strong acids, e.g. those below pH 2.0, or strong bases, e.g., those above pH 12.0, or organic solvents e.g., methanol, ethanol, acetonitrile, acetone, CS₂, dimethylformamide, dimethylsulfoxide, or the like. The bottom portion 12 of each of the sample wells is shaped so as to continuously decrease the cross-sectional area near the bottom opening 14 of each sample well. In a preferred embodiment the bottom well portion is conical in shape so as to focus protein molecules into a bottom opening 14 of reduced area at the bottom of each sample well. Below the bottom opening 14 is a separation layer 30 that serves to separate the sample wells 4 from capture material 40. The separation layer 30 functions to retain selected first sample molecules either within the sample wells 4, or within the separation layer 30, while allowing selected second sample molecules to pass through the separation layer into contact capture material 40 where the second sample molecules are captured concentrated. In a preferred embodiment of the invention the separation layer is comprised of a gel layer such as a polyacrylamide gel. Such gels generally have from 1% to 24% polyacrylamide, and also have various amounts of cross linkers and polymerization initiators and are well known to those skilled in the art of protein separations. Further in preferred embodiments having an array of sample wells 4, a corresponding array of substantially identical separation layers 30 will be present, preferably disposed within a cartridge gel plate 32, where the array of separation layers 30 is contained within an array of substantially identical apertures 34 disposed on a cartridge gel plate 32. In general, gel plate 32 is formed by machining, molding or casting from a desired material, such as thermoplastic polymers (polyurethane, polypropylene, and the like). Such gel plates willl be electrically-insulating, flexible polymers, e.g., thermoset polymers, elastomers, or rubber materials. In general such flexible material offers good liquid-sealing properties, while also providing electrical isolation between sample wells 4. The separation layer 30 also serves to isolate the sample wells 4 from the one, or more, capture material 40 that serves to capture and concentrate analyte molecules that are electrophoretically driven through the separation layer 30. Advantageously separation layer 30 is covalently bound to plate 32. Such covalent attachment prevents loss of adhesion and facilitates assembly of the cartridge assembly. As mentioned above, a particularly useful separation layer for isolation of proteins in liquid media is polyacrlyamide. Thus, covalent attachment of polyacrylamide to its supporting structure surfaces is particularly useful. The chemical bonding of polyacrylamide to a solid polyacrylamide supporting structure serves both to form a physically strong composite structure and also to form tight liquid seal between the polyacryamide and the supporting structure. In the instant case the bond is formed between the polyacrylamide separation layer 30 and gel plate 32, specifically within the area defined by gel plate apertures 34. In a method to carry out such covalent attachment of polyacrylamide to its supporting surface, or surfaces, a polyacrlyamide reaction mixture is deposited within the gel plate apertures 34 within gel plate 32, followed by a chemical grafting step. A particularly robust and durable polyacrylamide separation layer 30 may be photografted to gel plate 32 by photographing according a basic two-step reaction sequence. Both reaction steps may be performed by using solutions containing monomers of acrylamide and bis acrylamide in contact with the supporting surfaces. Initiation of both polymerization (within the bulk sreaction mixture) and attachment of polyacrylamide to a surfaces of a supporting structure, e.g., the gel plate 32, is provided by using ultraviolet radiation or alternatively chemical initiators. Conveniently a physical retainer approximately the size of the gel plate may be used to retain both the gel plate and the reaction solutions containing the monomers. Further the reaction mixture may be retained in contact with the supporting structure by a thin sheet of material that is held in approximation with the supporting structure by physical means such a vacuum clamp.

In a preferred embodiment, first the solid surface to which polyacrylamide is to be attached is pretreated with a photografting reaction mixture. Subsequently, chemical grafting of polyacrylamide to the supporting surfaces and polymerization of the bulk polyacrylamide mixture may be prerformed simultaneously. For example, the grafting and polymerization reactions both may be initiated by UV-irradiation in situ. In a preferred mode, a presoaking step is employed that comprises adsorption of a photoinitiator to the gel plate material prior to the polymerization. The presoaking step, for example may comprise substeps of a) emoloying a presoak solution containing a type II photoinitiator followed by b) drying of the gel plate, for example in dry gas such as air. Alternatively the gas may be heated to employ drying.

Type II photoinitiators are commercially available, such as from Sigma-Aldrich Company. Generally, type II photoinitiators undergo a biomolecular where the excited state of the photoinitiator interacts with a second molecule (a coninitiator) to generate free radicals. Examples include benzophenones/amines thioxanthones/amines.

A particular example of a type II photoinitiator presoak solution is 0.006% (by mass) thioxanthen-9-one in methanol. In the preferred mode the attachment and polymerization processes discussed above are carried out by placing a reaction mixture onto the surfaces, forming a low- to no-oxygen environment by vacuum sealing the mold, and irradiating the mixture with UV energy for a time sufficient to generate copolymer molecules which are covalently bound to the interior surface of the wells. With ordinary sources of UV energy (such as a 5000-EC unit from Dymax Corporation Torrington, Conn., USA fitted with a H-lamp) generally the irradiation time will be between 1 second and 1 hour. Alternatively, with very intense sources of UV, or flash sources, the irradiation time may be very brief, e.g., from 1 microsecond to 1 second, or less. Still other suitable sources of UV irradiation include mercury arc lamps.

Additional types of materials suitable for use as polyacrylamide supporting structures to which polyacrylamide can be chemically bonded additionally include polyurethane, santoprene, polypropylene, and the like. In general any polymeric material containing abstractable hydrogen atoms at its surface, i.e., in its backbone or side-chain moieties, will be a suitable polymeric material for carrying out the subject invention. By way of example, the abstractable hydrogen may be in the form of a double-allylic hydrogen, an allylic hydrogen, a tertiary hydrogen, or a secondary hydrogen. Specific examples include but are not limited to polymers made from or containing polyolefins, hydrogenated polystyrene, cyclic olefin copolymer, poly(ethyleneterephthalate), nylon, polycarbonate, poly(vinyl chloride), polybutylmethacrylate, polystyrene, poly(dimethyl siloxane), or poly(methyl methacrylate). Additional photoinitiators for initiaing the bonding process generally include type II photoinitiators, well known to those skilled in the art, that have the property of partitioning to the surface of the solid polymer to be grafted (rather than into the bulk polyacrlyamide polymerization reaction mixture).

In a preferred mode the combined photografting and bulk polymerization mixture consists of approximately 68.7% volume aqueous buffering solution, 30% volume of a 40% (w/v) solution of acrylamide/N,N′-Methylenebisacrylamide present in a 19:1 ratio) in deionized water, 0.69% volume of a 0.6% (w/v) Thioxanthen-9-one in methanol, 0.41% volume of a 1% (w/v) ammonium persulfate in deionized water and 0.20% volume of 1,2-Di(dimethylamino)ethane (TEMED) (or EDMA). The reaction mixture is mixed and placed into a shallow container having a glass or polymer bottom surface. Particularly useful as such bottom surfaces are “non-stick” surfaces, e.g., Teflon®. The “nonstick” surfaces act to facilitate release of the polyacrylamide from the bottom surface following the bonding of polyacrylamide to its desired solid supporting structure contained within the container, e.g., the gel plate 32. In the procedure, the gel plate is placed into the combined photografting and bulk polymerization mixture and covered with a UV-transparent cover, such as UV-transparent glass or a thin polymer plate such that gel plate apertures 34 contain the reaction mixture while air bubbles are excluded. The construct comprising a UV-transparent plate, polyacrylamide reaction mixture and bonding supporting structure mechanically are held in place by binder clips, a vacuum clamp, or other suitable clamping means. A photomask may be used over the UV-transparent plate covering the gel plate such that only desired portions of the reaction mixture and supporting structures are illuminated by the UV light source. Thereby the polyacrylamide may be bound to the its supporting surfaces in a predetermined pattern. For initiation of photopolymerization the construct is placed into proximity of a UV irradiation device and irradiated for a suitable time, depending upon the wavelength and intensity of the irradiation. The time of irradiation is dependent on system factors, but is generally less than four minutes where the irradiation flux is 150 mW/cm² of irradiated surface area. Sufficient UV radiation is provided for example by a 5000-EC unit from Dymax Corporation Torrington, Conn., USA using a D-lamp operating at a distance of approximately 20 cm from the gel plate surface. After UV irradiation, the UV-transparent cover is removed and the polyacrylamide, being chemically bonded to a solid supporting structure (e.g., gel plate 32) by photografting, is removed from the container and rinsed with a suitable rinse solvent, such as deionized water, in order to remove any nonpolymerized reaction mixture. The resulting polyacrylamide/supporting structure unitary part (e.g., polyacrylamide gel bound to the gel plate) then is placed in an appropriate liquid medium, or sealed package for storage, or immediately is assembled into a cartridge for use. Supported polyacrylamide gels made in situ show excellent mechanical stability and good adhesion to the supporting material. The simultaneous polymerization process described above is particularly convenient to carry out so as to manufacture such chemically-bonding supported acrylamide structures in time-efficient manner.

Optionally, the polyacrylamide reaction mixture may contain contain additional useful ligands, for example, proteins, polysaccharides, DNA, RNA, or the like. Such ligands conveniently may be added to the polyarylamide reaction mixture, prior to polymerization. Alternatively, the ligands may be added to the polyacrylamide after polymerization, either by allowing sufficient time for diffucsion of the ligands from an adjacent soaking solution, or by active electrophoresis from the soaking solution inot the attached polyacrlyamide. For example, if desired, a modified carbohydrate material may be added to the polyacrylamide for the purpose of enhancing the retention of albumin by the polyacrylamide. Examples of such materials include blue dextran, protein-affinity modified silicas, or other materials that are known to those skilled in the art to bind albumin.

Capture Slides

The capture material 40 is disposed at orifices 50 located in cartridge capture slides 42, having a top surface 41 and a bottom surface 43. The orifices 50 have a top opening 52, at top surface 41 and a bottom opening 54 at bottom surface 43. The apertures also comprise internal wall surfaces 56 of capture slides 42. Capture material 40 is attached to the capture slides 42, generally at internal wall surfaces 56 of the orifices 50. The attachment is effected by a bonding means which may including welding, either by solvent, thermal, sonic or other welding means. Alternatively the capture material 40 may be attached to the capture slides 42 at the orifices 50 by means of covalent chemical bonding employing epoxy, methacrylate, cyanoacrylate, or other types of chemical bonding materials and resins.

In a preferred embodiment of a cartridge capture slide 42 containing 96 orifices 50 (also referred to as apertures) for holding capture material 40, the cartridge capture slide 42 is between about 4 and about 6 mm in length, between about 3 and about 4 mm in width, and about 1 mm in thickness. More preferably, the cartidge capture slide 42 is about 5.3 mm long, about 3.5 mm wide, and about 1 mm thick. Also preferably, the orifices 50 are substantially circular and are about 0.5 to about 1.0 mm in diameter. Similar dimensions apply to the preferred cartridge gel plate 32.

As shown in FIGS. 2 and 3, under the cartridge capture slide 42 is an electrolyte base chamber 60 that functions to physically isolate and electrically connect the individual cartridge wells from each other and also from one, or more, common counter electrodes 70 in corresponding one, or more, counter-electrode chambers 72. When ready for use, electrolyte base chamber 60 is filled with a conductive electrolyte base medium 62 and counter-electrode chambers 72 are filled with a counter-electrode electrolyte 74. The base medium and counter-electrode electrolytes are in ionic communication so as to electrically connect the capture material 40 in the capture slides 42 with the counter electrodes 70 in counter electrode chambers 72. The counter electrode chambers have side walls 76 that, when ready for use, are at least partially vertical over substantially their entire surface, so as to provide a continuous upward path for the escape of any gas bubbles (e.g. hydrogen or oxygen) generated by the action of electrode 70 on electrolyte 74. Advantageously, the electrolyte base medium 62 will be highly conductive, for example containing a universal purpose soluble anion and cation pair of from 0.001 to 1 molar concentration in aqueous solution. The universal purpose anion and cation pair may be substantially any soluble anion and cation pair that is compatible with the materials of comprising chambers 60 and 72, e.g. salts of sodium, lithium, calcium, magnesium etc. and of chloride, fluoride, sulfate, thiocyanide and the like. One preferred salt comprising the pair is KCl since the anion and cation have substantially identical diffusion coefficients, thereby minimizing any diffusion potential at an interface between any two electrolyte solutions having different concentrations of the electrolyte. In general, the universal purpose soluble anion and cation pair will not be either a weak acid or a weak base, since migration of the charged form of either the acid or base at an interface between any two electrolyte solutions having different concentrations of the weak acid or base, or different conductivity, would cause a change in pH at, or across, the interface. These electrolytes, however, may contain such weak acids or bases, judiciously selected and employed with a protocol to cause regulation or modification in electrolyte pH, as is described elsewhere herein.

Electrolyte base medium 62 may be provided as a gel; so as to increase its viscosity and prevent leakage, or trapping of air bubbles; by dissolving of gelling materials such as starch or agarose, or copolymerization of hydrophilic polymers, e.g. acrylamide or hydroxymethylmethacrylate, as is well-known to those skilled in the art. The one, or more, counter electrode chambers 72 may also be filled with electrolyte 74 having the same composition employed in electrolyte base chamber 60. Because chambers 74 are physically isolated from capture materials 40, however, a much wider latitude in selection of conductive salts comprising electrolyte 74 is possible. For example, high concentrations of inorganic salts (e.g. from 0.1 to 10 molar) and the general use of salts of either a weak acids or a weak bases, in order to provide for pH-buffering of the hydrogen or hydroxide ions produced by common counter electrodes 70, optionally may comprise counter-electrode electrolyte 74. Examples are 1.0 M pH 8.0 tris(hydroxymethyl)aminomethane-chloride (Tris) chloride, or 1.0 M, pH 9.2 potassium borate, or 1.0 M, 1.0 M, pH 7.0 imidazolium chloride, or the like, but virtually any suitable highly-buffered buffer solution would suffice, as well-known to those skilled in the art.

In a preferred embodiment, the electrolyte base chamber 60 of cartridge 2 will be pre-filled with a gelled counter-electrode buffer solution 74. For example, the gelled solution may be a 1% agarose gel, also comprising 1.0 M KCl, 1 mM histidine, pH 7.8. Also, in a preferred embodiment the separation layer(s) 30 in cartridge gel plate(s) 32 and the porous capture materials 40 in the cartridge capture slide(s) 42, of cartridge 2 will be pre-filled with ionically-conductive liquid media. For example the separation layer 30 may be polyacrylamide gel containing from 2% to 12%, or as much at 15% polyacrylamide polymerized in an electrolyte solution containing from 1 mM to 500 mM inorgainic salts. In one embodiment the electrolyte pre-filled in the separation layer will be 50 mM KCl, 100 mM histidine, pH 7.8. The composition of the electrolyte pre-filled into the porous capture materials 40 in the cartridge capture slide(s) 42, of cartridge 2, may be of a wide variety of conductive salts dissolved in a solvent. The solvent may be an aqueous liquid, or another suitable organic solvent, such as methanol, ethanol, propanol, or the like, or alternatively acetonitrile or any other water-soluble organic solvent. Optimally the solvent employed will also contain from 1 to 1 M organic or inorganic salts to provide suitable electrical conductivity through the porous capture materials electrolye. Conveniently, the same liquid solution used to form the separation layer, e.g. 10 mM KCl, 100 mM histidine, pH 8.0 may be employed.

Electronic instrumentation and control components are utilized together with disposable cartridge 2 to provide an analysis system. An adjustable +/−300V voltage source (i.e. with an adjustable range of 600 volts) 100 can be used to provide the electrical field needed for electrophoresis. Such relatively low voltage sources are sufficient because the separation distance can be less than 1 centimeter, generally about 0.1 to 0.5 cm. Also, to monitor progress of separation and capture steps, current passing through each sample well 2 from sample electrode 20 to counter electrode 74 may be monitored separately. For example, 96 individual current meters may be used. Multiple current meters may be comprised of a single circuit for measuring current, but with a sample and hold circuit for reporting the current value (for example at a 1 Hz reporting frequency). In a preferred monde, the results are displayed graphically on a computer monitor. Alternatively an adjustable constant curreint source may be used in lieu of the voltage source. Usually the current source will supply from from 0-100 milliamps per sample well. More usually, the current source will supply from 0-10 milliamps. Advantageously a computer controlled selectable, current source/voltage source may be employed. A preferred selectable source, and methods of its operations, are disclosed in Provisional Patent Application No. 60/748,771, filed Dec. 8, 2005, the specification of which is incorporated herein in its entirety by reference.

Alternatively the electronic components needed to carry out the subject invention may be even simpler and may, for example, include just a direct current voltage source and an array of sample electrodes. Ain this alternative embodiment, an adjustable +/−100 volt voltage source (usually <25 volts) may be used to provide the electrical field needed for electrophoresis. In a 25-sample analysis system, for example, 25 current meters are used, each with a sample and hold circuit for reporting the current value (at a 1 Hz reporting frequency). If desired, the results may be displayed graphically on a computer monitor. Alternatively, and even more simply, the electrophoresis may be performed with the voltage source alone, i.e, without monitoring current, but running the electrophoresis either for a predetermined time, or alternatively, until a detectable (visual, chemical. or electrical) end point is achieved.

Suitable apparatus for performing the method described below includes: a +/−100 V power supply; a 25-channel, individually adjustable, array of potentiometers; an Agilent model 34970A data acquisition/switch; a 25-wel Lexan cartridge; and a laptop computer. The software for the Agilent data acquisition system may be configured to record voltage and current as a function of time for each of the 25 sample wells. An Applied Biosystems Voyager DF and 4700 model MALDI mass spectrometer may be used for mass analysis and quantification of analytes, including proteins and peptides.

Describing operation of the system:

1. A mixture of a fist and second groups of sample molecules are placed in a sample well 4;
2. Sample electrode 20 is brought in electrical communication with the sample in sample well 4;
3. The sample electrode 20 is energized with voltage source 100 causing a faradaic reaction (i.e. and oxidation or reduction reaction) to occur in sample well 20 thereby causing an ionic current 200 to pass from electrode 20, through sample well 2, through separation layer 30, through capture material 40, through electrolyte base medium 62, through counter electrode electrolyte in counter electrode chamber 72, and finally causing a faradaic oxidation or reduction reaction (opposite to that occurring at the sample electrode 20 in the sample well 4) to occur at counter electrode 70;
4. The ionic current 200 results in an electric field that results in first charged sample molecules to become electrophoretically driven through the separation layer 30 and concentrated onto the capture material 40 located at the orifices 50 on cartridge capture slides 42;
5. Second sample molecules at the same time do not pass through the separation layer 30 either by consequence of having either no, or the opposite electrical charge as the first sample molecules, or alternatively by consequence of the second molecules to becoming lodged within, or othrwise retarded by, separation layer 30.
6. After capture of the first sample molecules onto cartridge capture slide 42, the slides are removed from the cartridge well frame 6;
7. The cartridge capture slide 42 then is washed with deionized water, or other suitable solvent, to remove salts and other substances that may interfere with analysis, such as mass spectrometry analysis;
8. A MALDI matrix solution is applied to the capture material(s) 40 on the capture slide(s) 42 and allowed to dry.
9. The capture slide having the dried MALDI matrix affixed to capture material 40 is inserted into a MALDI mass spectrometer and the mass of the first analyte(s) are analyzed via MALDI-MS. For example, the mean and standard deviation of each (m/z) peak height, or peak area may be determined as a function of the amount of sample material applied to sample well 4, or the source of the sample material applied (for example samples taken from a group of humans sharing a common characteristics, medical symptoms, or diagnosis). (Here m refers to mass and z to unit electrical charge.)

For example, in steps 8 and 9 during analysis of such prepared MALDI capture slides, small droplets of MALDI matrix dissolved in a suitable solvent are added to the analyte capture regions. The solvent is allowed to dissolve the analytes and, as the solvent evaporates, the analytes become incorporated within MALDI matrix crystals that form on the top surface of the capture membrane. After allowing time, usually for 1 minute to 60 minutes, for evaporation of the solvent liquid and formation of MALDI matrix crystals, the sample plate is ready for introduction into a MALDI mass spectrometer. Upon insertion of the MALDI sample plate into a mass spectrometer, the MALDI matrix crystals are illuminated with an intense UV laser light pulse resulting in ionization of a fraction of the analyte molecules, as is well known to those skilled in the art of MALDI-MS.

Bt way of further example, polyacrylamide may be used as the separation layer 30, in Step 5. When polyacrylamide is so used, the acrylamide or bis acrylamide contained in the polyacrylamide may be of sufficiently high concentration, crosslinking and thickness so that only molecules less than a selected molecular weight (or specifically, m/z) are allowed to pass through the separation layer. In the special case where the selected molecular weight is about 30,000 Daltons for proteins, i.e., the LMW fraction of proteins, the separation layer may be used to remove the highly abundant proteins, larger than 30,00 Daltons, from biological tissues including soft tissues, such as brain, muscle, liver, lung, pancreas, ovary, testes, and particularly blood plasma and serum. For serum, for example, the separation layer may remove albumin, IgG, IgA, hemoglobin, haptoglobin, antitrypsin and transferin, which normally comprise about 95% of the total mass of proteins in this modified tissue. Alternatively, a non-sieving gel, such as 1% agarose, may be incorporated in the cartridge gel plate 32 to carry out a separation without removal of the high molecular weight proteins. After capture of the one, or more analytes on the one, or more capture materials 40 of the cartridge capture slides 42 a MALDI matrix is applied to the capture materials 40 and the materials analyzed for the combined first and second molecules by MALDI-mass spectrometry as described previously.

A preferred embodiment of the invention has an array of sample wells 4, each having a top opening 8, side walls 10 bottom opening 14, and contained within the cartridge well frame 6. The preferred embodiment also has a corresponding array of sample electrodes 20 and an array of separation layers 30, one for each sample well. Preferably the array of separation layers 30 will be contained as an array in a cartridge gel plate 32 where the holes in the gel plate are spaced at appropriate pitch so as to align with the bottom openings 14 of the sample wells 4. After the analysis the slides 42 containing the array of one, or more capture materials 40 are achievable for re-examination or verification at a later date.

The cartridge capture slides 42 having an array of sample wells 4 may be present as a single capture slide, or as a stack of two, or more, cartridge capture slides stacked in series, where analytes pass serially through each capture material 40 present in the two, or more capture slides. When present as such a stack of two, or more, capture slides the capture material in each slide may be substantially identical, or alternatively, substantially different. Advantageously, the substantially different capture materials, in the successive serial capture sides may be used to fractionate different analytes into selected capture slides, as is described in more detail below.

As an example, the capture material 40 within capture slides 42 may be a single material, e.g., it may be made from porous poly(vinylidene difluorde (PVDF) obtained as Immobilon-P or ImmobilonP^{SQ} obtained from Millipore Corp., Billerica, Mass. (USA). The porous PVDF capture material may be attached to the capture slides 42 to form the capture layer 40 by either thermal, ultrasonic, or laser welding, as described in greater detail in U.S. patent application Ser. No. 10/963,336, filed Oct. 12, 2004. An extensive literature exists on the use of PVDF to capture proteins by electro blotting from polyacrylamide gels for analysis by MALDI-MS^{5,6,7,8}. Also, advantageously, coating such membranes with a thin layer of conductive material prevents electrically charging of such PVDF membranes during analysis by MALDI-MS⁹.
⁵Schreiner, M, K. Strupat, F. Lottspeich and K. Eckerskorn (1996) Ultraviolet Matrix Assisted Laser Desorption ionization-Mass Spectrometry of Electroblotted Proteins, Electrophoresis, 17, 954-961.

⁶Bienvenut, W. V., J. C. Sanchez, A. Karmime, V, Rouge, K. Rose, P. A. Binz and D. F. Hochstrasser (1999) Toward a Clinical Molecular Scanner for Proteome Research: Parallel Protein Chemical Processing before and during Western Blot, Anal. Chem. 11, 4800-4807.

⁷Lion, N., V. Gobry, H. Jensen, J. S. Rossier, H. Girault (2002) Integration of a Membrane-Based Desalting Step in a Microfabricatd Disposable Polymer Injector for Mass Spectrometric Protein Analysis, Electrophoresis 23, 3483-3588.

⁸Muller, M., F. Gras, P. A. Binz, D. F. Hochstrasser and R. D. Appel (2002) Molecular Scanner Experiment with Human Plasma: Improving Protein Identification by using Intensity Distributions of Matching Peptide Masses, Proteomics 2, 1413-1425.

⁹Scherl, A., C. G. Zimmermann-Ivol, J. D. Dio, A. R. Vaezzadeh, P. A. Binz, M. Amez-Droz, R. Cochard, J. C. Sanchez, M. Gluckmann and D. F. Hochstrasser (2005) Gold Coating of Non-Conducting Membranes before Matrix-Assisted Laser Desorption/Ionization Tandem Mass Spectrometric Analysis Prevents Charging Effect, Rapid Commun. Mass Spectrom. 19, 605-610.

Fractionation of sample analytes may be increased further by increasing the number of successive layers of the capture slides 42 to two, or more, as shown in FIG. 2. In this embodiment the cartridge capture slides 42 are stacked so that analyte molecules sequentially pass through capture material 40 of each cartridge capture slide. Fractionation of molecules of PP within the successive capture materials of the capture slides 42 may be further improved considerably by employing capture materials 40 of substantially different chemical or physical surface properties in each of the two, or more, successive layers of capture slides 42 such that each will have a substantially different affinity for structurally different molecules of PP (i.e., proteins and polypetides) in the sample.

In order to perform fractionation of sample proteins on multiple successive layers of capture slides 42, each capture slide may have a capture material 40 comprising a membrane. Thus in operation of the device, sample analytes, e.g., proteins or polypeptides, are electrophoretically driven sequentially through the two, or more, capture membranes. Advantageously, each capture membrane employed in sequence will have a substantially different affinity for different classes of analytes. Examples of such membranes with different affinities include PVDF, or other porous polymer, membranes coated with modifying materials, of lower molecular weight, that alter the affinity of the membrane for analytes. For example, hydrophobic membranes may be coated with graded concentrations of hydrophilic polymers and then performing a reaction step to irreversibly bind the hydrophilic polymers to the higher molecular weight membrane material. For example, porous PVDF membranes (e.g., Immobilon-P and Immobilon-P^{SQ} obtained from Millipore Corp., Billericia, Mass.) may be coated with different solutions, wherein each of the different solutions contains a different concentration of a neutral hydrophilic polymer. Examples of such lower molecular weight polymers include:
1. Polyethylene glycol (PEG), e.g. Fluka Cat. No. 94646, M. Wt. 35,000
2. Polyvinylpyrrolidone (PVP), e.g. Sigma Cat. No. PVP40T, M. Wt. 40,000
3. Polyvinyl alcohol (PVA), e.g. Sigma Cat. No. P8136, M. Wt. 30,000

Protocols for coating and irreversible binding of such low molecular weight polymers to such higher molecular weight polymeric membranes are well known in the prior art. An example method is described in a U.S. patent to coat and irreversibly bind highly charged polymers, such as Nafion.® to PVDF membranes¹⁰. This method employs baking of the coated membranes at a temperature below the melting temperature of PVDF to irreversibly bind the lower molecular weight polymers to the higher molecular weight PVDF. This method, although straightforward, leaves a substantial fraction of the coating polymer non-covalently.—bound to the membrane. This loosely-bound coating material subsequently suppresses analyte ionization during MALDI-MS analysis. Advantagewously, a variety of chemical cross-linking reagents, such as glutaraldehyde, may be used to covalently bind the polymers to the membranes irreversibly. For example the cross-linking reagents may be hetero or homo-bifunctional cross-linking reagents, as is well known in the prior art.
¹⁰Moya, W. (2002) Surface Modified Porous Membrane and Process, U.S. Pat. No. 6,354,443.

After performing the coating and irreversible binding, procedures for electrophoretic mobility-based fractionation may be optimized. Experiments performed have shown that small highly charged peptides and proteins are captured first onto a PVDF-based capture membrane. By progressively extending the separation time (or, alternatively, increasing the applied voltage) progressively larger proteins are captured onto a PVDF-based capture membrane. These experiments also have demonstrated that some of the captured peptides can be eluted from the capture membrane with organic solvents (or MALDI matrix solutions containing organic solvents) and detected quantitatively by MALDI mass spectrometry. Also, successive fractions of proteins found in the serum samples can be captured onto the membrane targets. The fractionation procedure may be optimized by the following method:

### Optimization Method

1. Apply a standard measured volume of a standard protein sample (for example as 2 μL standard human serum sample, or any other suitable standard mixture of one, or more proteins or polypeptides) by pipeting the same measured volume into each of the cartridge wells.
2. Apply an electrical field perpendicular to the plane of the membrane, by passing the current transversely though the membrane for a predetermined run time. For example a sufficient electrical voltage is applied so that a current density of 0.1 to 10 mA per sq. mm of membrane area passes through each well, for a run time in the range of from 5 to 120 minutes. During the time electrical field is applied, the current passing through each of the capture materials 40 may be monitored or plotted to ensure uniformity and reproducibility in the electrical field in the capture materials 40 present at different sites in an array of capture materials disposed upon a multi-well capture slide 42. The current passing through the membrane causes electro-concentration of charged sample analytes within the capture material.
3. Remove the capture slide(s) from the PPAS cartridge after the electro-concentration procedure is complete.
4. Wash the capture slide free of salts or other interfering substances.
5. Apply a MALDI matrix solution to the capture material(s) 40 on the capture slide(s) 42 and allow to dry.
6. Insert the capture slide into a MALDI mass spectrometer and analyze via MALDI-MS. For example, the mean and standard deviation of each peak height may be determined as a function of the amount of serum sample used.
7. Perform optimization by repeating Step 1 through Step 6 at least two, or more times, each time varying either the current density, the run time, or both the current density and the run time. Generally the current density will be from 0.1 to 10 milliamp per square mm of membrane current density (or, more generally from 0.1 to 100 miliamps per square mm of apertures 50 in capture slides 42) and the run time will be between 5 and 120 minutes. The conditions (current density and run time) which give either the greatest number of protein or polypeptides peaks as detected by a mass spectrometer from the standard sample, or the greatest intensity for any one, or more, peaks are then adopted as the “standard optimized condition.”
The optimization method may be performed with biological samples, such as normal human serum (100 mL) purchased from Sigma Chemical Company, or an equivalent commercial source. Alternatively, such biological samples may be other biological fluids such as plasma, urine, cerebrospinal fluid, ascites fluid, saliva, or the like. Other suitable biological samples include lysed cells, either from biological tissues or obtained from cell culture. The optimization method may be repeated one, or more times sequentially while varying in sequence one, or more additional parameters; such as sample composition (e.g., pH and conductivity) and volume, electrolyte buffer composition, time, current density, capture materials, MALDI matrix solution composition or buffer or sample volume. The data obtained by MALDI-MS in the optimization method is analyzed and compared the results and the experimental parameters correlated so as to optimize the number and height of PP analyte peaks distinguished in the mass spectra.

In one embodiment of the above method, the current-voltage relationship during application of the electrical field in Step #2 is measured as a function of time. From the current-voltage relationship the change in resistance through the capture material 40 is calculated over time in order to determine when to terminate Step #2. For example, a time-course may be performed to capture fractions of different electrophoretic mobility on the array of capture membranes for discrete time periods encompassing 5-minute intervals from 5 minutes to 45 minutes. The resulting data is analyzed to determine the efficacy for time based LMW human serum fractionation. This time-base fractionation is then used as a protocol for analysis of serum peptides and proteins in selected ranges of molecular weights. The first fractions target peptides of about 1-2,000 Daltons, successive fractions may be in the 2-5,5-10, 10-15, 15-25, 25-50, 50, 100, 100-200 and >200 thousand Daltons. An associated standard operating protocol (SOP) for analysis of each molecular weight range may be selected such that a single sample may be analyzed in each of the molecular weight ranges and the spectra combined to provide for a complete proteome profile.

Prior to performing the analysis and optimization the serum is divided into aliquots of from 10 microliters to 10 ml, e.g. 450 μL aliquots, and stored at −80° degrees centigrade. For example, the following experiments may be performed to demonstrate pH-based LMW serum sample fractionation with the PPAS. The sensitivity and reproducibility of the apparatus for detection of peptide/protein standards in sample buffer (and also with the standards spiked into normal human serum) ma be examined at any selected pH value where the analytes are stable. For example with proteins and peptides conveniently pH 7.0, as well as pH values from 3 to 11 may be employed. Suitable buffering species are selected to buffer in each one of the desired pH ranges. (The buffering species are not critical because the capture membrane does not have appreciable ion-exchange properties and because unbound buffer species are washed from the capture membrane prior to detection by MALDI mass spectrometry.) Initial peptide/protein standards used for this purpose may be ubiquitin, gramicidin, cytochrome C, insulin oxidized B Chain and ACTH fragment (18-39). Additional suitable standard proteins may be added for each range of protein molecular weight applications to be covered by the PPAS. The sensitivity of detection for each of the standards in human serum (as defined as 3 times the standard deviation above the noise) may be determined. Approximately 20 PPAS cartridges may be analyzed to determine reproducibility of the system. Half of the cartridges may be processed in negative electro-concentration mode (i.e., negatively charged analytes are electrophoretically driven from sample wells 4 and concentrated onto capture materials 40) and the other half in the positive mode (i.e., positively charged analytes are electrophoretically driven from sample wells 4 and concentrated onto capture materials 40). The generated methods may be used to fractionate each of the samples into 5 or more fractions.

Alternative to using a preformed membrane material, such as PVDF, for the capture material 40, a substantially similar-functioning capture material may be cast into orifices 50 in the capture slides 42. For example, the capture material may be a hydrophobic porous polymethacrylate, such as poly(butylmethacrylate), poly(methylmethacrylate) poly(ethylene-dimethacrylate) poly(benzylmethacrylate, or mixtures of these polymers, such as poly(butylmethacrylate-co-ethylene-dimethacrylate). Alternatively, the capture material may be a hydrophilic porous polymethacrylate, such as poly(2-hydroxyethylmethacrylate), poly(glycidylmethacrylate), poly(diethylene glycol dimethacrylate), or mixtures, thereof. Still more advantageously the capture material may be formed from a mixture of hydrophilic and hydrophobic polymers, such that the hydrophobicity may be precisely selected from a range of hydrophobicities. The cast polymers may be deposited and attached to the sidewalls 56 of the orifices 50 in capture slides 42 according to a multiplicity of procedures well known to those skilled in the art
¹¹Lämmerhofer, M., F. Svec, J. M. J. Fréchet and W. Lindner (2000) Monolithic Stationary Phases for Enantioselective Capillary Electrochromatography, J. Microcolum Separations, 12, 597-602.

¹²Lämmerhofer, M., F. Svec, J. M. J. Fréchet and W. Lindner (2001) Capillary Electrochromatorgrapy in Anion-Exchange and Normal-Phase Mode using Monolithic Stationary Phases. J. Chromatography A 925, 265-277.

¹³Rohr, T., M., D. F. Ogletree, F. Svec and J. M. J. Fréchet (2003) Surface Functionalization of Thermoplastic Polymers for the Fabrication of Microfluidic Devices by Photinitiated Grafting, Advanced Functional Materials 13, 264-270.

¹⁴ Rohr, T., M., E. F. Hilder, J. Donovan, F. Svec and J. M. J. Fréchet (2003) Photografting and the Control of Surface Chemistry in Three-Dimensional Porous Polymer Monoliths, Macromolecules 36, 1677-1684.

¹⁵ Stachowiak, T. B., T. Rohr, M., E. F. Hilder, D. S. Peterson, J. F. Svec, M. Yi and J. M. J. Fréchet (2003) Fabrication of Porous Polymer Monoliths Covalently Attached to the Walls of Channels in Plastic Microdevices, Electrophorisis 24, 3689-3693.

¹⁶Peterson, D. S., T. Rohr, J. F. Svec, M. Yi and J. M. J. Fréchet (2003) Dual-Function Microanalytical Device by In Situ Photolighographic Grafting of Porous Polymer Monolith: Integrating Solid-Phase Extraction and Enzymatic Digestion for Peptide Mass Mapping, Analytical Chemistry 75, 5328-5335.

¹⁷Peterson, D. S., Q. Luo, M., E. F. Hilder, F. Svec, and J. M. J. Fréchet (2004) Porous Polymer Monolith for Surface-Enhanced Laser Desorption/Ionization Time-of Flight Mass Spectrometry for Small Molecules, Rapid Commun. Mass Spectrom. 18, 1504-1512.

¹⁸Pucci, V., M. A. Raggi, F. Svec, and J. M. J. Fréchet (2004) Monolithic Columns with a Gradient of Functionalities Prepared via Photoinitiated Grafting for Separations using Capillary Electrochromatography, J. Sep. Sci. 27, 779-788.

¹⁹Lee, D., F. Svec, and J. M. J. Fréchet (2004) PhotoPolymerized Monolithic Capillary Columns for Rapid Micro-High-Performance Liquid Chromatographic Separation of Proteins, J. Chromatography A, 1051, 53-60.

²⁰Hilder, E. F., F. Svec, and J. M. J. Fréchet (2004) Development and Application of Polymeric Monolithic Stationary Phases for Capillary Electrochromatography, J. Chromatography A, 1044, 3-22.

In a preferred embodiment of manufacture of capture slides 42, the side walls 56 of the orifices 50 in the capture slides are first vinylized to enable covalent attachment of the porous monolith polymer to the walls 56. In the vinylization procedure the orifices 50 first are rinsed with acetone then with deionized water; activated with a 0.2 mol/L sodium hydroxide for 30 min, washed with water, followed by 0.2 mol/L HCl for 30 min; and finally, rinsed with ethanol. Next a methacrylate polymerization mixture comprising 20% solution of 3-(trimethoxysilyl)propyl methacrylate in 95% ethanol with its pH adjusted to 5 using acetic acid is flushed through the 1 mm deep monolith for 30 min. Following washing with ethanol and drying in a stream of nitrogen, the functionalized slides are left at room temperature for 24 hours. Next, the orifices are carefully overfilled filled with the methacrylate polymerization mixture. The mixture contains methacrylate monomers and also porogen solvents, as disclosed^{11,12,13,14,15,16,17,18,19,20,21,22}
¹¹Lämmerhofer, M., F. Svec, J. M. J. Fréchet and W. Lindner (2000) Monolithic Stationary Phases for Enantioselective Capillary Electrochromatography, J. Microcolum Separations, 12, 597-602.

¹²Lämmerhofer, M., F. Svec, J. M. J. Fréchet and W. Lindner (2001) Capillary Electrochromatorgrapy in Anion-Exchange and Normal-Phase Mode using Monolithic Stationary Phases. J. Chromatography A 925, 265-277.

¹³Rohr, T., M., D. F. Ogletree, F. Svec and J. M. J. Fréchet (2003) Surface Functionalization of Thermoplastic Polymers for the Fabrication of Microfluidic Devices by Photinitiated Grafting, Advanced Functional Materials 13, 264-270.

¹⁴Rohr, T., M., E. F. Hilder, J. Donovan, F. Svec and J. M. J. Fréchet (2003) Photografting and the Control of Surface Chemistry in Three-Dimensional Porous Polymer Monoliths, Macromolecules 36, 1677-1684.

¹⁵Stachowiak, T. B., T. Rohr, M., E. F. Hilder, D. S. Peterson, J. F. Svec, M. Yi and J. M. J. Fréchet (2003) Fabrication of Porous Polymer Monoliths Covalently Attached to the Walls of Channels in Plastic Microdevices, Electrophorisis 24, 3689-3693.

¹⁶Peterson, D. S., T. Rohr, J. F. Svec, M. Yi and J. M. J. Fréchet (2003) Dual-Function Microanalytical Device by In Situ Photolighographic Grafting of Porous Polymer Monolith: Integrating Solid-Phase Extraction and Enzymatic Digestion for Peptide Mass Mapping, Analytical Chemistry 75, 5328-5335.

¹⁷Peterson, D. S., Q. Luo, M., E. F. Hilder, F. Svec, and J. M. J. Fréchet (2004) Porous Polymer Monolith for Surface-Enhanced Laser Desorption/Ionization Time-of Flight Mass Spectrometry for Small Molecules, Rapid Commun. Mass Spectrom. 18, 1504-1512.

¹⁸Pucci, V., M. A. Raggi, F. Svec, and J. M. J. Fréchet (2004) Monolithic Columns with a Gradient of Functionalities Prepared via Photoinitiated Grafting for Separations using Capillary Electrochromatography, J. Sep. Sci. 27, 779-788.

¹⁹Lee, D., F. Svec, and J. M. J. Fréchet (2004) PhotoPolymerized Monolithic Capillary Columns for Rapid Micro-High-Performance Liquid Chromatographic Separation of Proteins, J. Chromatography A, 1051, 53-60.

²⁰Hilder, E. F., F. Svec, and J. M. J. Fréchet (2004) Development and Application of Polymeric Monolithic Stationary Phases for Capillary Electrochromatography, J. Chromatography A, 1044, 3-22.

²¹Peterson, D. S., T. Rohr, J. F. Svec and J. M. J. Fréchet (2002) Enzymatic Microreactor-on-a-Chip: Protein Mapping Using Trypsin Immobilized on Porous Polymer Monoliths Molded in Channels of Microfluidic Devices, Analytical Chemistry 74, 4081-4088.

²²Svec, F., (2004) Preparation and HPLC Applications of Rigid Macroporous Organic Polymer Monoliths, J. Sep. Sci., 27, 747-766.

Incorporation of hydrophobic monomers into the polymerization mixture permits hydrophobic monoliths to be manufactured. Similarly, selection of hydrophilic monomers allows hydrophilic monoliths to be manufactured. Also, mixtures of hydrophilic and hydrophobic monomers at any predetermined ratio may be employed to manufacture monoliths of any desired hydrophilicity or hydrophobicity. In any of these cases, a xenon lamp fitted with a water filter (to remove infrared radiation) may be used to initiate the polymerization. While employing a xenon lamp of 150 watts, or greater, polymerization is completed after about 10 min of irradiation at a distance of about 10 cm. After polymerization, the solvent acting as a porogen in the polymerization mixture is washed away, for example by using a pressurized flow of methanol delivered with a syringe pump. Alternatively, porogens may be removed by simple diffusion into a rinse solution over a period of 12 hours, or more. Porous monolithic polymers, offer several advantages compared to polymers composed of small beads. For example, the monolithic polymers permit a significant increase the active surface area. Also, the monolithic polymers permit direct attachment to the walls of orifices 50.

Subsequent to the steps of fractionation and capture, each of the layers in the cartridge capture slide 42 may be disassembled and analyzed separately in a mass spectrometer as described herein. The additional fractionation into the two, or more, capture layers provides both more information about the proteins (indicated by the nature of the affinity incorporated into each capture membrane) and also provides increased sensitivity of detection by MS (because each capture material has proportionately fewer PP total molecules and thus a greater fraction of substantially identical PP molecules may be incorporated into each capture material 40.

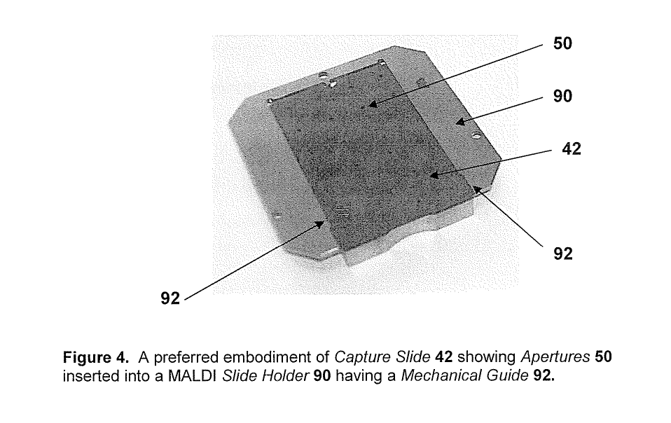

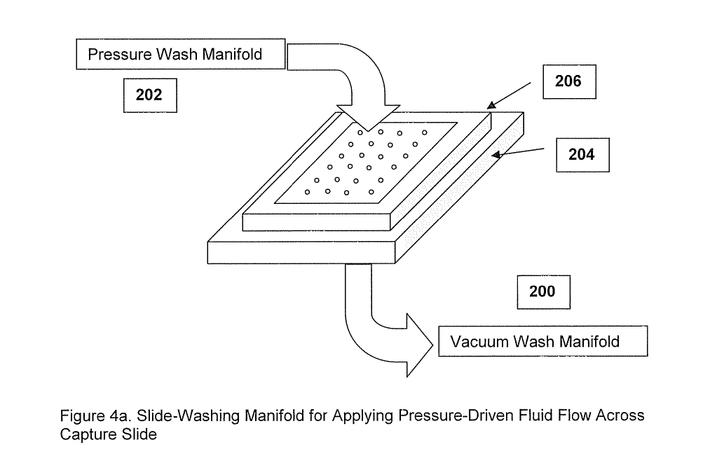

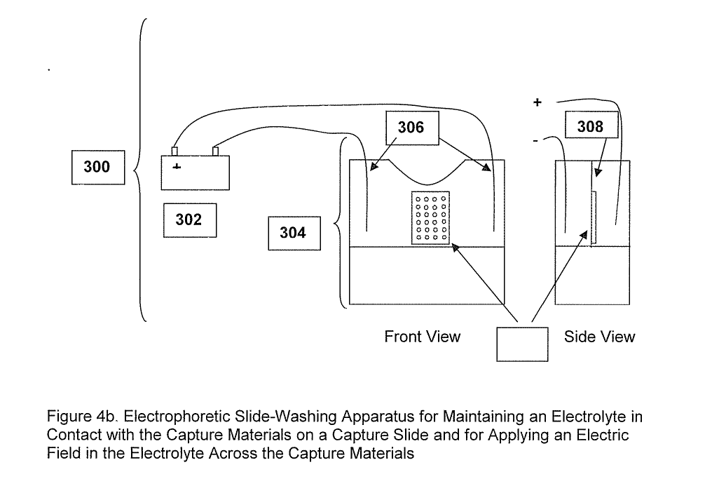

FIG. 4 shows a preferred embodiment of a cartridge capture slide that may be inserted directly into a standard slide holder 90 for an Applied Biosystems, Inc./Sciex Voyager DE MALDI TOF mass spectrometer. The cartridge capture slides 42 are made from a low electrical conductivity material so that greater than 10% of the electrophoretic current passes through the electrolyte in the apertures 50 also containing the capture material 40 in the cartridge capture slides 42. More usually the conductivity of the cartridge capture slides will be such that 75% to 99.999% of the current passes through the electrolyte in the apertures 50 also containing the capture material 40 in the cartridge capture slides 42. In order to achieve this during operation of the device usually the volume resistivity of the material used to make the cartridge capture slides 42 will be between 10² and 10¹⁰ ohm-cm. More usually the volume resistivity of the cartridge capture slides 42 will be between 10⁴ and 10⁶ ohm-cm. This slight conductivity of the materials, however, prevents charging of the capture slide 42 during ionization of the captured analytes in subsequent analysis by MALDI-MS analysis. Advantageously the cartridge capture slide 42 also is very flat, or alternatively may be designed to be flattened by insertion into a MALDI slide holder 90, such as that shown n FIG. 4, to +/−50 microns. The cartridge capture slide 42 may be attached to sample holder 90 by means of a mechanical guide 92, or alternatively by a ferromagnetic material, such as a magnet. For example, the magnet may be a small rare-earth magnet, e.g., a neodymium-iron-boron (NdFeB) magnet about 1 mm in thickness and about 2 mm in diameter. The ferromagnetic material functions to hold the lower component frame member (and the attached capture membrane) to a MALDI sample plate during MS analysis of sample analytes on the capture membrane. For this purpose, these magnets clamp with sufficient force to (#318 stainless steel).

In operation, electrically-charged mobile analytes migrate in the applied electrical field from the sample well 4, which may be in the array of multiple sample wells, disposed in a cartridge well frame 6 of an analysis system. Each sample well 4 serves to retain an electrolyte fluid comprising a sample having one, or more sample analytes (e.g., PPs). Each sample well also serves to accept a sample electrode 20 that is inserted into the electrolyte fluid to establish electrical contact with the fluid. When a voltage is applied to the electrode 20 with respect to a common counter electrode 70, an ionic current flows through the fluid in well 4, comprising sample and a pH-buffered electrolyte diluting solution, thereby creating of an electric field in the sample well 4. The electric field results in electrophoretic movement and separation of the one, or more, analytes in the sample well 4. Advantageously, the apertures containing the capture material are substantially smaller in cross-sectional area than that of the sample wells 2, so as to provide for electrophorectic concentration of analytes within the capture material 40 within apertures 50. A preferred embodiment of the analysis system includes sample wells 4 that accommodate sample volumes of from 1 to 400 μL. The inside diameter of each well is approximately 6.7 mm at top opening 8 and narrows to approximately 1.0 mm at its bottom opening 14 so as to permit concentration of analyte molecules by electrophoresis into a narrower diameter aperture 50 containing a capture material 40) in capture slide 42. The wells narrow within a bottom portion 12 of the interior side walls 10 of the wells. Generally a side wall in such bottom portion 12 will have a slope between 20 and 30 degrees from the, generally vertical, center axis of wells 2. In a preferred embodiment the slope will be between 24 and 26 degrees from the center axis of wells 2. Generally, the diameter of the sample wells 2 will be between 5-20 mm and the capture region diameter between 10 microns and 1.5 mm.

Separating the bottom opening 14 of sample wells 2 from capture slide 42 is a thin separation layer 30. The separation layer may be comprised of sieving material (e.g. polyacrylamide gel) that is filled with electrolyte to maintain the two regions in ionically conductive and fluidic contact. The sieving material may be pre-cast and assembled, or cast in place, If cast in place, the polyacrylamide layer may be made by pouring liquid acrylamide monomer and cross-linker into the wells to any desired thickness. The liquid then is allowed to polymerize prior to assembly, for example either by incorporation of a free-radical chain-initiator such a ammonium persulfate, or by the addition of a photo-sensitizer, such as riboflavin, and illumination with light of a wavelength absorbed by the photo-sensitizer, e.g. either UV light or 400-450 nm light for riboflavin. Further, the separation layer 30 may be provided as, one or more, serially stackable sieving or separation layers. For, example, an agarose gel may be used in series combination with either a porous polyacrylamide layer, a porous dialysis membrane, or both. When in such a series combination, the porous agarose advantageously acts a first pre-filter to keep the polyacrylamide layer from becoming overloaded with either sample analytes or interfering substances such as high abundance proteins. The polyacrylamide, in turn, acts as a second pre-filter filter to keep the dialysis membrane from becoming clogged with protein during the electrophoretic concentration.

Generally the capture material 40 will be contained in apertures 50 that pass transversely through cartridge capture slides 42. The charged analytes that pass through the separation layer, driven by electrophoresis, then are captured in the capture material 40 of an assembly on one, or more, cartridge capture slides (CCS) 42 the assembly constructed so that the orifices of successive cartridge capture slides align, coaxially, so that an analyte may pass sequentially through the porous capture material 40 in each aperture. Thereby such captured analytes are concentrated from a larger volume of the sample well 4 into a smaller volume in the capture material 40 retained in the capture slides 42. Also multiple analytes may be separated captured by the capture material in the successive cartridge capture slides, thereby substantially fractionating the analytes into different slide fractions. The assembly of cartridge capture slides 42 comprises one, or more, sequential capture slides, usually from 1-10 such sequential slides, more usually from 1-5 sequential slides, but potentially from 1-100, or more, sequential slides as a series of stacked layers. The stack of sequential layers of capture slides 42 is constructed so that during operation ionic current is made to pass serially through each layer of slides 42 from a first sequential capture slide, then a second sequential capture slide, and so on until passing through the last sequential capture slide.

Advantageously, the capture material 40 in the apertures 50 of the capture slides may contain a modified capture material, where the modification increases the affinity of capture material 40 for selected analytes. Further such modified capture slides may be modified to have differential high affinity for different analytes. Still further, such modified capture slides with differential high affinity for different analytes may be stacked sequentially so that analytes encounter a first capture slide, then a second capture slide, then a third, and so one, each capture slide 42 having a capture material 40 with differential high affinity for different analytes. The first sequential capture slide 42 may have a high affinity for a first selected analyte. The second sequential capture slide 42 may have a high affinity for a second selected analyte. Further, capture material 40 in a third selected sequential capture slide 42 may be selected to have a high affinity for a third selected analyte, and so on, in sequence. Thereby the first, second and third, analytes may be captured specifically by the first, second and third sequential capture slides 42. Thereby fractionation of the first, second and third analytes into the sequential capture slides may be performed conveniently and rapidly.

The analytes having a high affinity for the selected capture slides for the may be predetermined. For example, any analyte that is a member of an analyte-anti-analyte binding pair, where a capture material 40 is modified by attachment of the anti-analyte, the capture material 40 of a predetermined capture slide 42 will result in specific capture of the predetermined analyte in the predetermined slide. For example, an analyte may have an antigenic epitope recognizable by an antibody such that immobilization of that specific antibody to the capture material 40 in a predetermined sequential capture slide 42 will result in specific capture of the predetermined analyte in the predetermined slide 42. In lieu of the antibody any complementary member of a binding pair may be utilized to bind the complementary analyte. Such complementary members herein are called ligand-receptor pairs. The affinity of liqand-receptor binding may be selected to have a high affinity or low affinity. Different analytes may be captured selectively by sequential capture slides having different affinities for different analytes. Thereby fractionation of the analytes into the separate layers can be achieved.

Each capture material 40 is attached to a cartridge capture slide 42 comprising a rigid solid support thereby facilitating subsequent manipulations of the capture material including washing, drying, application of a MALDI matrix, a second drying step, and mass analysis in a MALDI mass spectrometer. Multiple capture materials, with the same, or different affinity for different analytes, thus can be inserted into the apertures 50 in multiple capture slides, stacked serially so that the apertures align, one with the other so that analytes pass sequentially through the different capture materials. For example, each of the slides may consist of a polypropylene frame having one, or more, small orifices with a porous polymer membrane, or monolith cast, welded, glued, or otherwise attached to each of the one, or more, orifices comprising capture regions.

The capture material 40 in the apertures 50 of capture slides 42 is in (ionic) electrical and fluidic communication with the top 44 and bottom 46 surfaces of the cartridge capture slide 42 and thus will carry electrical current through it. Electrical contact to the bottom surface of the capture slide 42 is made through an electrolyte base medium 62 that is comprised of an ionically-conductive (electrolyte) medium (such as an agarose gel) contained in electrolyte base chamber 60. The electrolyte base medium makes electrical contact with common counter electrode 70 through a counter electrode electrolyte contained in the counter electrode chamber 72 which houses the common counter electrode. The system is constructed so that when an electrical voltage is applied between a sample electrode 20 and the common counter electrode 70, an electrical current flows between the two electrodes. The current is carried by ionic species in the electrolytes disposed between the electrodes. Thus charged analyte present in the sample well are electrophoretically driven either towards electrode 20 or counter electrode 70. The analytes driven toward electrode 70 are concentrated in the capture material 40 present in the electrical path when a voltage of predetermined polarity is applied between sample electrode 20 and counter electrode 70. Applying the selected voltage polarity to two, or more of the sample electrodes, with respect to a counter electrode causes analytes from the two, or more, of sample wells to be concentrated into two or more corresponding capture materials in a capture slide 42, separately, and simultaneously. The voltage applied to the sample wells may be selected to be of either both positive or both negative polarity. Thus, either positively charged or negatively charged analytes may be concentrated into the separate capture materials, either individually, or simultaneously. Alternatively, the sample electrode polarity may be predetermined to be positive in one well and negative in another well, thus capturing negatively charged and positively charged analytes in two, or more different capture materials in a single capture slide 42 simultaneously. In the analytical system 300, individual electrical circuits are connected from the sample electrodes through a sample well, through a separation layer, through an aperture in the capture slide 42, through the electrolyte base chamber and through the counter electrode electrolyte 72 contained in the counter electrode chamber and in contact with common counter electrode 70. Advantageously, the analysis steps may include dissociation and separation steps that result in depletion of high abundance analyte molecules from low abundance analyte molecules. Such steps are useful for highly sensitive and reproducible analysis of peptides and proteins analytes by mass spectrometry.

Such dissociation and separation steps may be performed more efficiently by employing the addition of a non-ionic detergent or other suitable dissociating agent to samples present in the sample wells 4. For example, the detergent may be added in a suitable pH-buffered electrolyte prior to the step of applying a voltage. Alternatively, the detergent may be added either to the samples, or any other reagent present within the sample wells 4. The nonionic detergent effectively dissociates hydrophobic peptides from large molecular weight, high abundance molecules such as albumin and IgG. Next, when a voltage (and resulting electrical current) is applied between the sample and the common counter electrodes analytes of selected charge in the sample are driven toward either the anode or the cathode (depending upon the sign of the applied voltage and the sign of the electrical charge on the analyte).

At any selected pH value of the sample, a binary separation of positively-charged and negatively-charge analytes may be performed. For example, operation at a sample pH of 7.8, and applying a positive voltage to sample electrode 20 with respect to the common counter electrode), will result in a positive current flowing from the sample electrode 20 to the common counter electrode 70. The positive current will cause positively charged analytes in the corresponding sample well 4 to migrate from well 4 and to be captured (on capture slide 42) in the capture material 40 present in the aperture 50 immediately below the corresponding well 4 (i.e., the device is said to be operated in the positive mode). Conversely when a negative voltage is applied to the sample electrode, a negative current will flow from sample electrode 20 to the common counter electrode 70. The negative current will cause negatively-charged analytes in the sample to migrate from the sample well 4 and to be captured (on capture slide 42) in the capture material 40 present in the aperture 50 immediately below the corresponding well 4 (i.e., the device is said to be operated in the negative mode). In either negative or positive mode the current carried from each sample electrode will usually be from 10 microamperes to 10 milliamperes. More usually the current will be between 0.2 and 2.0 milliamperes.

Customarily, at least two sample wells are used for fractionation of any one sample. In one of the sample wells, the sample electrode is polarized positive and in the other negative with respect to a common counter electrode. That is, the positive mode and the negative mode separations are carried out simultaneously. Separation of positively charged and negatively charged analytes then will occur simultaneously at a sample pH predetermined by the system operator. Thus fractionation of sample analytes into those positively-charged and negatively charged at the predetermined pH may be performed simultaneously. Further, separation of a single sample into two, or more, fractions of different isoelectric point is possible by employing sample buffer solutions in any two, or more, sample wells having two different of pH values. Thereby fractionation, concentration, and capture of analytes according to isoelectric point may be accomplished as is disclosed below in further embodiments of the invention.

In addition to charge-based fractionation according to isoelectric point, a sieving material optionally may be employed in the separation layer between the sample and the capture material. Analytes electrophoretically driven from a sample well 4 towards a corresponding capture material 40 first must pass through the sieving separation layer 30. The sieving layer thus serves to provide for additional fractionation by retarding the migration of high molecular wt. analytes with a given m/z value with respect to lower molecular wt. analytes with the same m/z values, as is well know to those skilled in the art of gel electrophoresis. (The migration time of proteins through polyacrylaimide gel is well known to be approximately proportional to the logarithm of molecular weight of the proteins, when a detergent (e.g., sodium dodecylsulfate) is present to bind to the proteins roughly in proportion to molecular weight and thus give all proteins a similar m/z value.) Thereby the sieving material may be used to isolate the LMW proteome fraction when the value of m/z is similar for proteins of different molecular size. In such separation by sieving, the time of application of the predetermined voltage, or current, is chosen to provide for optimal separation of proteins in any predetermined range of molecular weight. Lower molecular weight (i.e., higher mobility) analytes will pass through the sieving layer more quickly and thus will be captured on the capture materials 40 prior to the lower mobility analytes. Thus a kinetic separation may be performed by the subject invention. Such high mobility analytes either may be concentrated into a single capture material or a further separation performed in combination, by passing through a series of two, or more, stackable capture slides, where each of the two or more slides have at least one aperture coaxially aligned with other apertures of an adjacent capture slide. Also, in sequence, each aperture may have a different predetermined capture material, thereby performing separation of analytes based upon affinity and thereby providing for maximal fractionation with a minimum number of capture slides. For example the different capture materials may comprise a difference in hydrophobicity of the capture materials. By way of further example, the top capture material positioned closest to a sample well) may be the least hydrophobic and the capture material sequentially farthest from the sample well may be the most hydrophobic. Thereby a gradient of hydrophobicity is created in order to provide for separation and analysis of analytes according to their hydrophobicity. With such fractionation by sequential slides performed by affinity, then when the system is operated in combination with a sieving separation layer 30, both molecular weight and affinity separations are performed in combination and simultaneously in combination. Since a multiplicity of two, or more samples may be separated independently and simultaneously with in cartridge 2, multiple samples may be separated by multiple such modes, simultaneously.

Advantageously the fractionation and capture steps can be carried out relatively quickly provided that the separation and capture layers are relatively thin. For this purpose the separation and capture layers usually will be between 20 microns and 20 mm in thickness. More usually the separation and capture layers will be between 200 microns and 5 mm in thickness. For thin separation and capture layers, for example from 500 microns to 2.0 mm in thickness, the fractionation steps may take from 10 seconds to 100 minutes. Customarily the separation and capture will occur in less than 1 hour. More usually the separation and capture will be performed in between 1 minute and approximately 10 minutes. After the capture step, the PPAS device is disassembled (as shown in FIG. 3) and each of the cartridge capture slides is briefly washed to remove salts or other chemical species that interfere with detection by MALDI or electrospray mass spectrometry. For example the capture slides simply may be rinsed in deionized water. After the washing step, a MALDI matrix solution is applied to each of the capture regions of each capture slide and matrix allowed to dry. After the drying step, the slides are directly inserted into a mass spectrometer (e.g. a MALDI-TOF MS) for mass analysis of the captured analytes. Alternative, to detection of captured analytes directly from the capture material 40 in a mass spectrometer, the analytes may first be eluted from the capture material and detected by any variety of means, including MALDI-MS, electrospray, mass spectroscopy, proteolytic digestion by enzymes, e.g. trypsin, and analysis of the resulting peptide fragments, i.e. by constructing a “peptide map,” or any other analysis means as are well know to those skilled in the art of protein identification and analysis. The cartridge capture slides may be injection molded from carbon-doped polypropylene to permit direct MALDI analysis without charge spreading.^{i,ii,iii,iv,v} The capture material 40 may be formed from a hydrophobic membrane such as polyvinylidine difluoride (PVDF) attached to the capture slide material by any suitable means, for example by using an adhesive, or by welding through application of a solvent or heat to either the capture material, the slide, or both. Alternatively the capture material may be cast into orifices in the capture slides. For example, the capture material may be porous poly(butyl methacrylate-co-ethylene dimethacrylate) polymer monoliths. Such monoliths may be cast by polymerization according to procedures published by Svec et al.^{vi,vii,viii,ix,xi,xii} For robust capture slides having tightly bound capture material, the internal wall surface of the slide orifice is first vinylized to enable covalent attachment of the monolith to the walls.^{xiii} Orifices are rinsed with acetone and water; activated with a 0.2 mol/L sodium hydroxide for 30 minutes, washed briefly with deionized water, followed by 0.2 mol/L HCl for 30 min; and finally, rinsed briefly with ethanol. A 20% solution of 3-(trimethoxysilyl)propyl methacrylate in 95% ethanol, pH 5 (for example ethanol with to 0.1 to 1.0% acetic acid) is flushed through an approximately 1 mm thickness monolith for about 30 min. Following washing with ethanol and drying in a stream of nitrogen, the functionalized slides are left at room temperature for about 24 h. Proper choice of monomer permits selection of monolith hydrophilicity. Next, the orifices are carefully overfilled filled with the methacrylate polymerization mixture, covered to prevent evaporation and allowed to polymerize. Standardly, a Xenon lamp fitted with a water filter is used to initiate photopolymerization. Polymerization is completed after about 10 min of irradiation at a distance of about 10 cm. The monoliths then are washed for about 12 hours by using methanol delivered by a syringe pump, or any other suitable means of providing a relatively slow and continuous flow. Porous monolithic polymers, compared to polymers composed of small beads, permit a significant increase the active surface area. Alternatively, as disclosed more fully below, as a preferred embodiment, mixtures of such porous monolithic polymers together with chromatography particles may be used as a capture material 40.
ⁱKnochenmuss, R. Anal. Chem. 2004; 76: 3179.

ⁱⁱZalluzec, E. J.; et. al. J. Am. Soc. Mass Spectrom. 1994; 5: 230.

ⁱⁱⁱAndrews, P. C.; et. al. Anal. Chem. 1996; 68: 1910.

^{iv}Andrews, P. C.; et. al. Electrophoresis, 1997; 18: 382.

^{v}Costello, C. E.; et. al. Rapid Commun. Mass Spectrom. 1999; 13: 1838.

^{vi}Peterson, D.; Rohr, T.; Svec, F.; Fre'chet, J. M. J. Anal. Chem. 2003; 75: 5328-5335.

^{vii}Fréchet, J. M. J.; et. al. Macromolecules 2003; 36: 1677-1684.

^{viii}Fréchet, J. M. J.; et. al. Journal of Chromatography A 2004; 1044: 3-22.

^{ix}Fréchet, J. M. J.; et. al. Electrophoresis 2003; 24: 3689-3693.

^{x}Fréchet, J. M. J.; et. al. Journal of Chromatography A 2004; 1051: 53-60.

^{xi}Svec, F. J. Sep. Sci. 2004; 27: 747-766.

^{xii}Fréchet, J. M. J.; et. al. Rapid Commun. Mass Spectrom. 2004; 18: 1504-1512.

^{xiii}Ericson, C.; Liao, J. L.; Nakazato, K.; Hjerte'n, S. J. Chromatogr., A 1997; 67: 33-41.

^{xiv}Tempst, P.; et. al. Anal. Chem. 2004; 76: 1560-1570.

For MALDI-MS analysis of sample analytes captured on capture materials 40 on the capture slides 42 within cartridge 2, as prepared within the PPAS device, small droplets of MALDI matrix dissolved in a suitable solvent are added to the analyte capture regions. The solvent is allowed to dissolve the analytes and as the solvent evaporates, the analytes become incorporated within MALDI matrix crystals that form on the top surface of the capture membrane. After allowing time for evaporation of the solvent liquid and formation of MALDI matrix crystals, the sample plate is ready for introduction into a MALDI mass spectrometer. As an example, FIG. 4 shows the cartridge capture slide and its direct insertion into a standard Applied Biosystems, Inc./Sciex Voyager DE MALDI TOF mass spectrometer slide holder. Upon insertion of the MALDI sample plate into a mass spectrometer, the MALDI matrix crystals are illuminated with an intense UV laser light pulse resulting in ionization of a fraction of the analyte molecules, as is well known to those skilled in the art of MALDI mass spectrometry.

Removal of Interfering Chemical Species from Capture Slides by Selective Washing Compositions and Methods

After capture of an analyte on capture material 40 retained within apertures 50 of capture slides 42, the capture slide is removed by disassembly of cartridge 2. Next, salts and inorganic, or organic pH-buffering species selectively are washed free of the capture slide and capture material retained within the apertures of the slide. The washing compositions are carefully chosen however, to retain the analytes of interest on the capture material during the washing process. Such selective washing of hydrophobic capture materials to retain PP analytes, such a proteins and peptides, customarily will utilize substantially aqueous solvents. Washing may be done, by a) diffusion, pressure-driven flow, electrophoresis (i.e., removal of electrically-charged interferants), or alternatively by electroendosmosis, or by a combination of any two, or more, such methods.

For example, pressure-driven flow of wash solution may be effected by a device, such as that shown in FIG. 4a designed to apply a differential pressure across the capture slide 42. Such a pressure differential may be applied, for example by applying a vacuum with a vacuum manifold 200 to one side of the slide causing fluid from a fluid bath on the opposite side to flow through the capture material 40 in the slide toward the vacuum manifold 200. Alternatively a positive pressure may be applied by means of a positive pressure manifold 202 to the side of the slide having the fluid bath, thereby effecting substantially the same pressure-driven flow of the washing fluid across the capture material. In either case, capture slide 42 is supported by a pressure-retaining support 204 working in conjunction with a slide sealing means 206, such as a rubber, or soft poymeric gasket, or “O-rings” to provide for sealing. Advantageously the vacuum or positive pressure may be used to apply fluid flow substantially simultaneously, across a multiplicity of two, or more, capture materials 40 within two, or more, apertures 50 within a capture slide 42. A fluid that may be used for the washing procedure, for example can be deionized water (DI) or alternatively a “MALDI-friendly” ion-containing solution such as 0.1% trifluoracetic acid (TFA) in DI to purge the interfering salt from the capture material 40 while retaining desired PPanalytes bound to the capture material. Such “MALDI-friendly” ions characteristically are those ions than, when neutralized by loss, or gain of a proton, have an appreciable vapor pressure and can be “pumped off” rapidly in the vacuum chamber of a mass spectrometer. Examples of such “MALDI friendly” materials, that are ionic at selected pH values, are acetic acid, ammonia, formic acid, propionic acid, piperizine, pyridine etc., as is well known to those skilled in the art of preparing samples for MALDI-mass spectrometry.

Alternatively, as shown in FIG. 4b, an electrophoretic device 300 may be employed to apply an electric field across capture material 40 in slide 42. The electrophoretic device comprises voltage source 302, a fluid reservoir and slide holder 304 having an electode pair 306 to serve as an anode and cathode, and a septum 308 acting to isolate the anode from the cathode so that current must pass through the apertures 50 within capture slides 42.

For most effective electrophoretic washing of capture materials on capture slides free of inorganic salts and inorganic and organic pH buffering species, the following principles are used, either singly, or in combination:

A. Hydrophobic Ion Exchange.

A first ion-exchange step is used to exchange MALDI-unfriendly interfering species for MALDI-firendly ions, which, when the pH is adjusted will have an appreciable vapor pressure, as discussed above. When the capture materials on capture slides have an affinity for hydrophobic ions, e.g. “reversed phase” chromatograpy priciples are used to capture analytes, hydrophobic interfering species will be bound to the capture materials as well. Accordingly in the first step, such hydrophobic interfereing species are exchanged for hydrophobic ions of like charge, (i.e. either positively charged, or negatively-charged species. For example if histidine buffer (isoelectric point 7.8) is employed, (zwitter ionic histidine is extremely “MALDI-unfriendly”) histidine may be exchanged for negatively charged trifluoractetate ions at a pH where histidine is negatively-charged, i.e. at a pH above 7.8. Customerily 0.1M trifluoracetic aciid at pH 9.0 is used for this purspose. Passing I milliamp of current thorugh 1 mm apertures Caputre Slide 42 for 5 minutes has been found to be particularly effective for this purpose. Other negatively-charge or positively-charged interfering species, such as the buffering species HEPES, TES, HEPPS, CAPS, CHES, ACES, ADA, BES, MES, MOPS PIPES can be removed individually, at a selected pH above their individual isoelectric points, by similarly exchanging these negatively p-charge ions (anions) for trifluoracetic acid anions. Alternatively, by way of example, histidine can be exchanged for positively-charged pyridine ions (cations) by performing the electrophoretic wash at a relatively low pH, i.e., at a pH<7.8 where both histidine and pyridine are positively-charged, e.g., at pH 4.0, for example.

B. High-Field Electrophoresis

By this method, the conductivity of the electrolyte is reduced by substantial dilution, for example in distilled water, and a large electrical field then is placed across the capture material 40 in capture slides 42. In this method the loosely-bound hydrophobic buffer ions dissociate from the monolith, they are swept out by the high field before they can rebind. In this method, the pH will be in the 3-11 range, and more usually for optimal performance, will be in the 4-10 range, so as to keep the conductivity relatively low. Low conductivity is required so as to apply a high electrical field without producing an excessively large current. With the devices disclosed above, currents above 1 millamp per well will cause excessive Joule heating within the apertures 50 of capture slides 42. Such Joule heating is well known in the prior art to be proportional to the square of the current, i.e. proportional to I²R, where I indicates the current and R the resistance through apertures 50).

C. Electroendosmotic (EEO) Flow

Flow generated by EEO is proportional to the electrical field across the monolith, and also is a function of the zeta potential (i.e., the potential drop across the plane of shear from the solid phase monolith to the liquid in the monoliths). As charged hydrophobic species are washed free of the capture materials 40, the zeta goes to zero. The flow, thus diminishes as the washing step is completed. A high electrical field is optimal for high EEO, thus substantially the same conditions optimal for High-Field Electrophoresis mentioned above will be optimal for EEO flow.

D. Coulombic Repulsion

This is mechanism is the simplest. Capture slides 42 are place into a dilute electrolyte, such as distilled, or deionized water at a pH where the bound buffer ions are charged. Coulombic repulsion of the ions pushes them out of the microliths. Optimally the ionic strength of the wash solution will be low, i.e., under 1 milimolar dissolved ions. Also any hydrophobic species that might ion pair with the interfering ionic species to be washed free of the capture material are to be avoided. For example, if positively-charged histidine is an interfering species (i.e. histidine is bound at a pH less than its PI of 7.8) then acetate, formate, TFA, HEPES, PiPES, or other hydrophobic anions would be avoided.

Example Electrophoretic Washing Procedure:

To remove from capture materials 40 any interfering hydrophobic anion e.g., TFA, HEPES, PiPES (in the neutral pH in the 4-10 range) the following steps are carried out:
1. A wash solution of 0.1% trifluoracetic acid, pH 9 (+/−0.5 pH units) is used to supply 1 milliamp per square mm of aperture area through apertures in the capture slides for 5 minutes. This will accomplish ion exchange.
2. After carrying out step #1, the wash solution is diluted approximately 1/100 with distilled, or deionized water and the voltage is increased to provide for about 0.125 millamps per square mm of aperture area (or the maximum available for the power source, whichever is less). This washing step is performed for another 5 minutes and will cause washing via principles B through D).

When interfering hydrophobic cations are present as interfering species bound to capture materials 40 the same above steps are carried out, but instead employing a MALDI-friendly cationic species, such as pyridinium is employed, instead of trifluoroacetic acid. Pyridinium trifluoroacetate at a pH between 4.0 and 5.0 is particularly effective.

In either case, the combination of the two above electro-washing steps is superior to either electro-washing step alone.

Trans-Elution of Captured Analytes from Cartridge Capture Slides and MALDI Matrix Addition for Analysis by MALDI Mass Spectrometry

Once analytes have been concentrated and captured onto the capture materials 40 retained with apertures 50 of cartridge capture slides 42, and potiential MALDI interfering species removed, captured analytes then may be analysed. For example, analysis by MALDI-TOF mass spectroscopy may be performed. Standard MALDI-MS matrix compositions and methods may be used to dissolve captured proteins and deposit them within MALDI matrix crystals for analysis in a MALDI mass spectrometer. Such standard procedures are well known in the field of mass spectrometry and have been well documented in the literature. (See for example, Cohen, S. and Chait, B. T. Anal. Chem. 1996, 68, 31-37.)

An example standard MALDI matrix and procedure is to employ a matrix solution consisting of a mixture of 1 part of 20 mg/ml sinapinic acid in acetonitrile and 1 part 0.1% (v/v) trifluoroacetic acid in water (i.e., the final concentration of sinapinic acid is 10 mg/ml). contained in a cartridge capture slide, 0.25 microliters of the mixture of matrix solution is carefully added to the sample side each capture material 40 so the majority of the solution remains on the material (rather than spreading to the surrounding slide material). After drying in air, a second, equal volume addition of the matrix solution is applied in the same manner. The cartridge capture slide 42 then is dried, either by air drying or by means of a vacuum applied within a desiccator. After removal of the acetonitrile and water solvents, MALDI-MS measurements and analysis may be performed in a MALDI-mss spectrometery. Conveneintly the capture slide 42 is inserted into a slide holder 90 having a mechanical guide 92 for retaining the slide. An example of such a slide holder adapted for use in Applied Biosystems Voyater MALDI mass spectrometers is show in FIG. 4. In such analysis by such a method optimal results are obtained by applying the MALDI matrix solution to the capture material exposed at the top surface 41 of the capture slide 42. Subsequently, the top surface 41 of slide 42 also is positioned in the sample holder of a mass spectrometer, so that for MALDI-mass spectrometry the same surface, 41, of the capture slide is probed with the laser beam of the MALDI-mass spectrometer, and therefore emits the analyte ions to be detected by the ionic current detector with the mass spectrometer.

In a preferred alternative analysis procedure advantageously an analyte elution solvent is first applied to the bottom surface 43 of the sample slide 42 (i.e., to the surface opposite that of the sample well 4). By this procedure analyte molecules are eluted from the capture material and concentrated at the top surface 41 of the capture slide prior to formation of (and incorporation of analyte molecules within) MALDI matrix crystals. This procedure makes the elution process more sensitive, decreases the analysis variation, and makes the analysis less dependent upon the depth within the capture material where an analyte is captured. The MALDI matrix may be applied, either to the bottom surface 43 of slide 42 together with the elution solvent, or alternatively to the top surface 41 after the analyte elution process is complete.

An example method is as follows:

1. A sample, or plurality of samples are is placed into sample wells 4 of cartridge 2.

2. A predetermined voltage, or a predetermined current is applied to each sample wells by sample electrodes 20.

3. Analyte molecules of predetermined charge are electrophoretically separated from other analytes though separation layer 30 and are concentrated and captured though the top surface 41 of a capture slide 42 at sites having a porous capture material 40.

4. The cartridge is disassembled so as to gain access to the capture slide 42.

5. Following a washing procedure to remove interfering species, anaytes are eluted from the porous capture material to an analysis side of the capture slide, which in a preferred mode is the top surface 41.

6. MALDI matrix is applied to the same analysis side of the slide, which in the preferred embodiment is the top surface 41.

7. The MALDI matrix is dried in air, other dry gas, or a vacuum and the capture slide is inserted into a MALDI mass spectrometer for analysis so the analysis surface is exposed to the laser beam probe and the ion detector of a mass spectrometer. In the preferred embodiment of the invention the top surface 41 of the capture slide is so exposed.

8. Analytes captured onto the capture slides are analysed for the mass, (more precisely thier m/z value) and their relative abundance.

In the preferred mode step #5 and #6 above are combined as follows:

The cartridge capture slide is inverted over a drying apparatus (such as a 1-10 cm/sec air velocity fan) and a solution of acetonitrile and deionized H₂O (typically 9:1 v/v) is applied to the capture material 40 exposed at bottom surface 43 of each capture slide 42. After allowing a few minutes for the elution solvent to be drawn through the porous capture material, this step is followed by a second elution step that includes MALDI matrix, e.g., concentrated sinapinic acid (e.g. 9.0-90 mM in deionized water, pH 7.0-8.0) in methanol (typically also 9:1: v/v). This step is followed by a third elution step wherein the pH is adjusted to be acidic (typically 9 parts of acetonitrile and 1 part of 0.1% trifluoracetic acid in deionized H₂O. To ensure that the MALDI matrix (e.g., sinapinic acid by means of vacuum drying in a desicator. After sufficient drying, MALDI-MS measurements and analyses are performed in a MALI-mass spectrometer such as a Bruker Autoflex model, or an Applied Biosystems Voyager model, for example. This method of MALDI matrix addition also provides for the elution of biomolecules to the topside of the slide, further reducing the limits of detection of analyte molecules in such MALDI-MS measurements.

Preferred Cartridge Capture Slide Configurations, Capture Materials and their Method of Manufacture

Cartridge capture slides 42, has apertures 50, and capture materials 40 residing within the orifices. In a preferred embodiment the capture slide has 96 apertures, disposed in a 8×12 rectangular array (i.e., 12 columns and 8 rows) wherein the center of each aperture is spaced apart 9.00 mm from each of the closest four neighboring apertures (i.e., has a 9.00 mm pitch). In the preferred embodiment, the apertures are approximately 1 mm in diameter and approximately 1 mm in depth. In manufacture, the generally flat capture slide, with very small variation in thickness (typically less than ca. +/−50 μm), has orifices that are formed by machining, (for example by laser, or mechanical drilling) molding, or casting, as is well know n to those skilled in the art of polymer device manufacture. In a preferred embodiment, the capture slide material is selected to optimize the bulk and surface conductivity. As mentioned previously, the conductivity of the cartridge capture slides 42 will be such that 75% to 99.999% of the current applied to the capture slides by sample electrodes 20 passes through the electrolyte within the apertures 50 rather than passing through the bulk slide material). In order to achieve this condition during operation of the device usually the volume resistivity of the material used to make the cartridge capture slides 42 will be between 10² and 10¹⁰ ohm-cm. More usually the volume resistivity of the material used to make the cartridge capture slides 42 will be between 10⁴ and 10⁸ ohm-cm. This conductivity of the capture slide material prevents charging of the capture slide 42 during ionization of the captured analytes in subsequent analysis by MALDI-MS analysis. Alternatively, the bulk conductivity of cartridge capture slides may be either more, or less conductive, and the surface conductivity is adjusted to achieve the desired condition by providing a resistive surface coating to provide the equivalent quantity of electrical conductivity.

Once the capture slide 42, with apertures 50, is formed, capture materials 40 may be deposited and attached within the apertures by a number of means, such as attachment of membranes by welding, either with solvents or by heating, casting of the materials into the apertures, or other means of attachment. In a preferred mode, casting is provided by performing grafting via two photopolymerization reactions in situ. Both reactions are performed in a mold on a vacuum table by using ultraviolet radiation to initiate photo polymerization. In the method a suitable mold for casting is formed from thermoplastic, thermo set, or metal by machining, or otherwise fashioning the mold. The mold must retain the capture materials 40 within apertures 50, of capture slides 42, and advantageously will exclude oxygen which acts to terminate free-radical polymerization reactions, as is well known to those skilled in the art. For example the mold may be comprised of a thin sheet of material such as polyethylene or “Saran Wrap” that is held in place against the slide apertures by vacuum while the slide is held on a vacuum table, as is well know to those skilled in the art of such molding procedures.

The first photo polymerization step double bonds, or vinyl groups are photo grafted, to the walls of apertures 50. In the photo grafting process a photografting solution is placed into the apertures and irradiated with UV light for a time necessary to generate copolymer molecules which are covalently bound to the capture slide material circumscribing apertures 50. When the UV irradiation is provided by a 5000-EC unit from Dymax Corporation Torrington, Conn., USA using an H-lamp, the irradiation time needed generally will be from 1-5 minutes in length.

A suitable photografting reaction mixture consists of 48.5 mass % methyl methacrylate (MMA), 48.5 mass % ethyleneglycol dimethacrylate (EDMA) and 3 mass % benzophenone. The reaction mixture is weighed, mixed and sparged with a gas such as argon, helium or nitrogen to drive out oxygen. The sparged reaction mixture is placed into the wells by dipping the capture slide into the mixture and then tapping to remove excess, by pipetting into each well, or by otherwise delivering the reaction solution to the interior of the well. The capture slide is then placed into the mold, the mold is placed on the vacuum table (Pharmacia Fine Chemicals, Model GSD-4) and the vacuum is turned on. A UV-transparent plastic sheet is then placed over the filled apertures the mold, Such plastic sheet can be provided from commercial plastic wrap such as Saran Wrap, from sheet rubber, such as polydimethylsiloxane sheet, or any suitable UV-transparent gas barrier material. The plastic sheet is manually held in place against the capture slide retained in the vacuum table until a sufficient vacuum develops to retain the slide. Following the photo grafting reaction is then placed into a UV irradiation device with a mercury arc lamp and irradiated for a time. The time of irradiation is dependent on system factors, but is generally less than one minute where the irradiation flux is 100 mW/cm2 of irradiated surface area. Sufficient UV radiation is provided for example by a 400 W Hg lamp operating at a distance of approximately 20 cm from the capture slide surface. After UV irradiation, the plastic cover is removed; the photo grafted capture slide is removed from the mold and rinsed with acetone. The photo grafted slide is then placed in acetone and stored there for a time to remove trace amounts of monomers and any segments of copolymer that may remain ungrafted to the surface of capture slides 42.

The second photografting step comprises in situ formation of a solid, but porous, monolith material that is in part attached to the capture slides 42 via the photografted copolymer attached in the previous step. In the present method, monolith formation is carried out by placing a reaction mixture into the wells, forming a low- to no-oxygen environment by vacuum sealing the mold, and irradiating with UV for a time to generate the monolith from a mixture of monomers and porogens. Such porogens are known in the art to promote the formation of porous solids when mixed with reactants that subsequently form a solid phase. The UV irradiation may be provided from an SLM instruments 400 Watt Xenon arc lamp, or alternatively, the UV irradiation is provided by a 5000-EC unit from Dymax Corporation Torrington, Conn., USA using a D-lamp.

In a preferred method, monolith “reaction mixture A” is used. “Reaction mixture A” comprises 5 grams 1-decanol, 2.4 grams n-butyl methacrylate, 1.6 grams EDMA, and 1 gram cyclohexanol along with an initiator. Dimethyl acetophenone (DMAP) is used as the initiator in 1% proportion to the total mass of monomer, thus in this case 0.4 grams DMAP is used. The reaction mixture is weighed, mixed until the DMAP is entirely dissolved and sparged with a gas such as argon, helium or nitrogen to drive out oxygen. The sparged reaction mixture is placed into the wells by first filling the mold with approximately 10 mL of reaction mixture, then placing the slide into the mold. Alternatively, the reaction mixture can be pipetted into each well or otherwise delivered to the interior of the well. The capture slide is then placed into the mold, the mold is placed on the vacuum table (Pharmacia Fine Chemicals, Model GSD-4) and the vacuum is turned on. A plastic sheet is then provided to cover the mold, with sufficient plastic sheet directly atop the capture slide. Such plastic sheet can be provided from commercial plastic wrap such as Saran wrap, from sheet rubber such as polydimethylsiloxane sheet, or any suitable covering material. The plastic sheet is then held in place by holding it down against the vacuum table until the vacuum develops sufficiently to fixture the mold and contained capture slide to the vacuum table. The capture slide part is then placed into a UV irradiation device with a xenon or metal halide arc lamp and irradiated for a time. The time of irradiation is dependent on system factors, but is generally less than four minutes where the irradiation flux is 150 mW/cm2 of irradiated surface area. Sufficient UV radiation is provided for example by a 400 W Xe lamp operating at a distance of approximately 20 cm from the capture slide surface (SLM Instruments, Champaign, Ill., USA). After UV irradiation, the plastic cover is removed; the monolith-filled capture slide is removed from the mold carefully and rinsed with methanol. The monolith-filled slide is then placed in about 10 volumes of methanol for 1-24 hours to allow methanol to displace the higher alcohols and remove residual unreacted monomer. Fresh methanol is used to wash each subsequent batch to ensure adequate cleaning.

Many suitable variations (of the both the method and to reaction mixture A) exist, as generally is known to those skilled in the art. References 11-22 show examples. Through experimentation we have found that capture materials 42 formed by a heterogeneous combination of two, or more, different capture materials are superior to pure monolithic capture materials when used alone. In general, the heterogeneous combination comprises solid, preformed, particulate chromatography media consisting of solid or porous core particles The particles may be so called “reverse phase” particulate chromatography media (i.e. hydrophobic particles, or alternatively may be cationic, or anionic “ion-exchange media,” Examples of such materials include high purity silica, protein-affinity modified silica, polymeric chromatography porous or solid beads or other solid particulate materials that are known to those skilled in the art of chromatography or in the manufacture of such materials. Alternatively a mixture, or alternating layers of two, or more such media may be used. In each case the particulate chromatography media are held in place by a suitable matrix which may be any suitable material which adheres well to the surface of capture slides 42 and also firmly traps the chromatography media in place. Conveniently, for example, the same compositions mentioned above and taught generally in references 11-22 may be used for such a matrix. Generally, a preferred “reverse phase” heterogeneous combination, for the capture of proteins and peptides from biological samples, is made between, so called “reverse phase” particulate chromatography media and a monolithic solid phase. monolithic material, such as that cited above (and generally in references 11-22).

In particular, a preferred embodiment for the capture of biological peptides and proteins comprises a mixture formed whereby 33% of the “reaction mixture A” is replaced with Alltech SPE Bulk Sorbent C8 (Alltech Associates, Deerfield Ill., Cat No 211504). The general procedure described above for employing pure reaction mixture A is employed with the mixture. Also, advantageously the particulate material helps to increase the viscosity of the reaction mixture containing monomers, cross linker and initiating reagents. The increase in viscosity helps to prevent leakage of the polymerization reaction mixture from the apertures during casting. Thereby the viscosity may be adjusted to a desired value by selecting a predetermined particulate compositon, generally ranging from 1% particulate to 99% particulate material. More generally the particulate material will be between 10% and 90%. Even more generally the particulate material will be in the 25 to 50% range. Further examples of particulate chromatography particles that may be used to manufacture preferred capture materials for capturing proteins and polypeptides are given in Table 1.

TABLE 1
“Microlith” capture chemistry

| Capture Mechanism | Examples |
|---|---|
| Normal Phase | Silica, alumina |
| Reverse Phase | C2-C18, polymeric resins, monoliths |
| Ion Exchange | SCX, SAX, WAX, WCX |
| Immobilized metal affinity | Ni, Fe, Ga |
| Antibody Capture | Protein G, Protein A, Streptavidin, |
| | Custom Antibodies |
| Small Molecule Affinity | Blue Sepharose/Dextran, |
| | Custom ligand libraries |

These chromatography materials are provided by a number of manufactures in bulk quantities, having particle sizes ranging from 0.2-500 um. Either porous or nonporous particles may be employed, although porous particles are preferred because of their greater binding capacity per unit volume. By way of further example, manufacturers include Agilent, Alltech, Applied Biosystems, Phenomonex, Supelco, and Waters. A preferred embodiment of the present invention comprises C8 reverse phase resins, specifically Alltech (part # 206250), bound together with methacrylate resin, as described herein as the capture material. These particle resins combinations demonstrate high utility for the capture of biological macromolecules, including proteins and peptides in particular, and carbohydrates, polysaccharides, and oligonucleotides more generally, and provide for their subsequent desorption/ionization by using MALDI mass spectrometry.

Such combinations of unpolymerized resins and prepolymerized particles when subsequently polymerized as a unit are called “microlith” compositions herein. Such microliths consist of preformed and customarily, commercially-available chromatography media held into a thin capture slide configuration by a MALDI-compatible resin. The specific composition of such microliths are predetermined a according to the composition of chromatography media chosen for embedding. Such compositions include but are not limited to normal phase, reverse phase, ion exchange, immobilized metal affinity, small molecule ligand affinity, including antibody-capture affinity and lectin-capture affinity chromatography media, to name a few examples. Other examples are well known to those skilled in the art of chromatographic separations of biomolecules and selection of commercially-available media for such purpose.

One unexpected characteristic of the mixture of porous monolithic material and particulate media is that the combination increases the porosity of the solid phase capture material. Thus, pressure-driven flow through microliths constructed according to the methods described herein is much greater than through “monolithic capture materials, as describe both in the available literature, and as described herein. Another unexpected advantage of the combination (i.e., mixture) of porous monolithic resin and prepolymerized particulate chromatography media is that the combination increases the tensile strength of the resulting capture material. Thus, (100%) pure porous monolithic capture monolithic materials (e.g., formed from 100% reaction mixture A) when cast into 1 mm diameter apertures tend to crack and lose adhesion to the capture slide surface when dried in a vacuum. In contrast, incorporation of 33% of Alltech SPE Bulk Sorbent C8, prevents such cracking and loss of adhesion. Such heterogeneous compositions, generally referred to as “microliths” are made by the above methods and are found to have superior mechanical strength, stability and have good adhesion in the capture slide surface. Further we have found such “microliths” further to have the capacity to capture proteins, peptides and other analyte molecules in electrophoretic devices. Further such microliths may be cast sufficiently flat (e.g., +/−50 microns to provide and excellent surface for subsequent analysis using matrix-assisted laser Desorption/ionization Mass spectroscopy (MALDI-MS).

Dissociation and Removal of High Abundance Proteins from Serum

A major problem with analyzing clinically important, low abundance peptides in blood, plasma, or serum is that high abundance proteins mask the appearance of low abundance proteins and peptides. Affinity removal of the most abundant proteins from blood, plasma or serum samples, however, has been hypothesized to also remove a significant number of low abundance, hydrophobic peptides. In a preferred embodiment of the separation and analysis method example, serum samples first were treated with MALDI-compatible detergents in order to promote dissociation prior to subsequent molecular weight fractionation to remove high abundance, high molecular weight proteins.

All samples were either applied to stainless steel sample plates or to disposable capture slides made of a flat polymeric material having an electrically-conductive surface. For example, 2 microliter (μl) sample volumes may be applied either directly as a droplet of solution, or electrophoretically captured on monolithic capture materials that, after drying, may be placed directly into a MALDI mass spectrometer. By way of further example, 0.5 μl of MALDI matrix solution may be pipetted onto the sample spots and allowed to dry. Proteins and peptides generally are analyzed with alphacyano-4-hydroxycinnamic acid (CHCA) employed as the MALDI matrix, since it generally provides the best signal to noise MALDI-mass spectrometry results for low molecular weight peptides and polypeptides from 1,000 to 15,000 Daltons (Da). The composition of the CHCA matrix solution may be as follows: CHCA is saturated in a mixture of 50% acetonitrile and aqueous 0.1% trifluoroacetic acid. All materials for the MALDI matrix solutions may be obtained from Sigma Chemical Co (St, Louis, Mo., USA). All MALDI-MS analyses may be performed with an ABI Voyager DE MALDI-TOF and an in-house designed QGEN_PR2 method. Typical spectrometer settings are: 20 kV accelerating voltage, 94.1% grid voltage, 0.050% guide wire voltage, 110 ns delay, 3000 laser setting, 64 scans averaged, 1.1e-6 torr, 511 low mass gate, negative ions off.

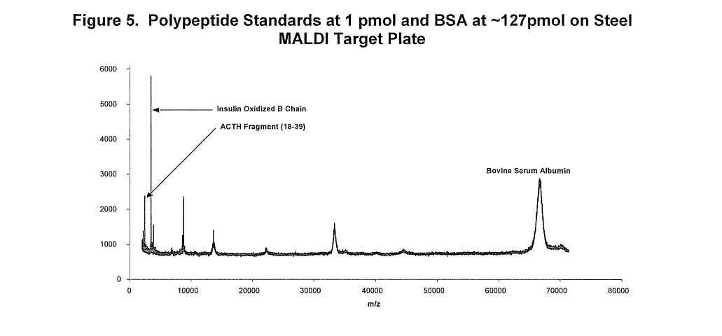

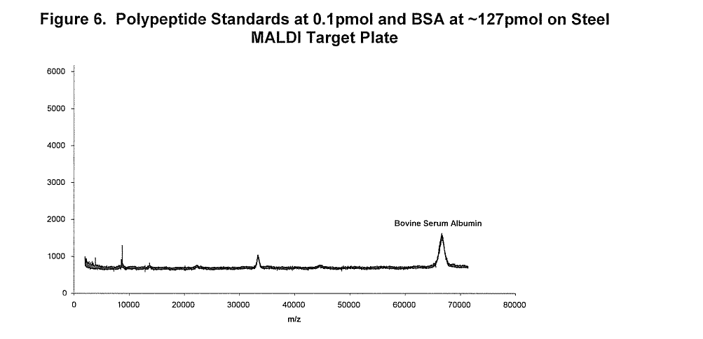

For demonstration two polypeptide standards, e.g. ACTH fragment (18-39), and insulin oxidized B chain, may be mixed together with bovine serum albumin (BSA) and pipetted directly onto a stainless steel MALDI target plate. FIG. 5 shows these two such polypeptide standards diluted to 1 picomol (pmol), while in the presence of ˜127 pmol BSA, and applied to replicate spots on a MALDI mass spectrometer plate and allowed to dry and then separately analyzed for molecular mass and intensity while in the presence of ˜127 pmol BSA. FIG. 5 clearly shows that the 1 pmol of ACTH fragment and 1 pmol of insulin can clearly be distinguished. Similarly, as shown in FIG. 6, when the amount of peptide fragments was 10-fold less, i.e., 0.1 pmol of the two standards, the same concentration and amount of BSA, substantially suppressed ionization during analysis by MALDI mass spectrometry as shown in FIGS. 5-7.

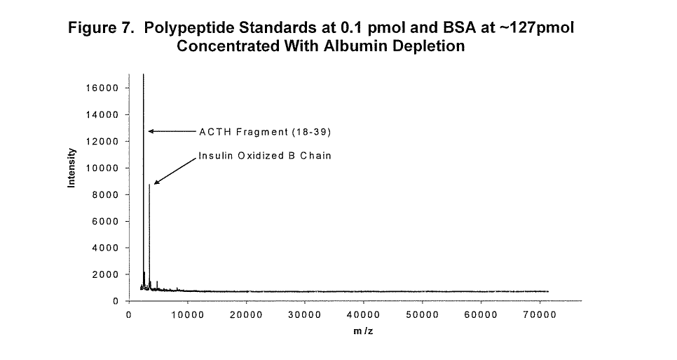

Shown in FIG. 7 is a MALDI-TOF spectrum of the same sample employed for the results seen in FIG. 6. For the results seen in FIG. 6, however, the sample was first prepared by electrophoresis, concentration and capture on a capture slide. The procedure entails using a single layer cartridge capture slide. In the procedure 2 μL of the sample was combined with 250 mM aqueous L-histidine buffer and processed by using a single monolith capture slide. Capture is allowed to occur by passing approximately 1 milliamp of current for a sufficient period of time so that the total charge transferred is approximately 1 coulomb. The results show that the system effectively removes the BSA as interference from the sample mixture. These results demonstrate that the device and protocols when used in combination effectively removes substantial signal interference from detection of lower molecular weight proteins and polypeptides (i.e, less than 30,000 Daltons) caused by larger proteins such as albumin, i.e., greater than 30,000 Daltons, thereby dramatically enhancing the mass spectrometry signal obtained from low molecular weight molecules.

LMW Human Serum Analysis

Experiments have been performed with embodiments of the invention by using a single cartridge capture slide. Such studies have been conducted to determine feasibility of preparation of human serum for low molecular weight protein/peptide profiling via MALDI MS according to protocols of the instant invention. For example, detergent-treated serum samples are made by adding 10 μg/μL octyl-b-D-glucopyranoside (OG) to 100 μL of human serum (obtained from Sigma Chemical Co.) in an Eppendorf microtube (500 μL volume). Samples are then made from 10 μL aliquot of the detergent-treated serum, 100 μL of 250 mM histidine buffer, 1 μL of Texas Red labeled-Leu Enkephalen (as a tracer in 250 mM histidine buffer) and O.5 μL of glycerol. The resulting sample mixtures then are centrifuged at about 1000 g for 1 minute in order to bring together the mixture droplets.

In order to perform separation and binding of sample components, a 10 μL aliquot of the prepared sample may be added to a sample well. For example, a cathode made of platinum may be placed directly into the sample well. The opposing electrode, a platinum anode iss placed in contact with counter electrode electrolyte place in a counter electrode chamber. The platinum anode and cathode electrodes are connected to a potentiostat (Princeton Applied Research, model 273) and approximately 1 mA of current applied between the electrodes. Separation was allowed to proceed for about 20 minutes before the voltage is set to zero and the leads to the electrodes disconnected.

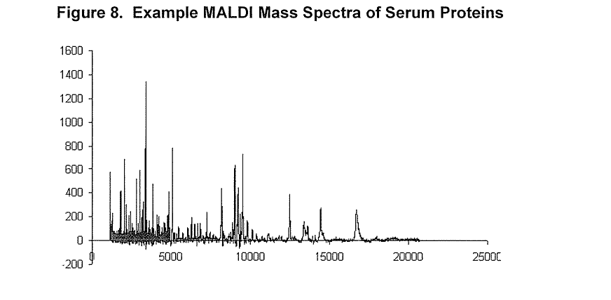

Next, the prototype cartridge is disassembled and the gels and capture layer checked for fluorescence. The analytical system is performing well when essentially all fluorescence from the proteins and peptides selected to bind to the capture materials 40 is observed to bind to the capture sites. The capture slides then are soaked in deionized water for approximately 5 minutes. After visual inspection of the fluorescent capture sites, the slide is allowed to air dry completely. Next, a 0.5 μL aliquot of MALDI matrix is applied to the topside of the capture spot. The capture spot is then analyzed directly in a Voyager DE MALDI MS. FIG. 8 shows the mass spectrum obtained from a sample by using CHCA as the MALDI matrix. The Figure shows good signal to noise ratios for the detection of low molecular weight polypeptides from human serum.

When using similar parameters described in the above example, blood serum may be applied to two wells within a PPAS cartridge. One, or more, of samples may be treated with a detergent to promote dissociation of proteins, on from the other. In so doing, detergent-treated samples may be combined with 250 μL of L-histidine, adjusted to pH 6.8 and current applied at 1.0 mA by means of polarizing a sample electrode in contact with the sample. The other detergent-treated samples may be combined with 250 μL of L-histidine, adjusted to pH 7.0 and similarly biased with a sample electrode to provide a current of −1.0 mA. As shown in the figures, the spectra observed from the two, oppositely-polarized sample wells show completely different, complementary protein and peptide peaks. These data clearly demonstrate the binary pH fractionation of the same sample.

Further Examples and Methods

Materials. All materials are available from commercially vendors and include: acetonitrile, trifluoroacetric acid (TFA), n-octoglucoside, CHCA, L-histidine and polyacrylimide. Serum preparations may be conducted in 0.5 mL polypropylene tubes from Simga Co. C-18 coated superparamagnetic beads may be purchased from Bruker Daltonics.

Serum Samples. Blood samples from volunteer subjects with no known malignancies and from consenting patients with confirmed prostate cancer (Gleason scores 6-7) may be provided in 8.5 mL glass Vacutainer tubes, allowed to clot at room temperature for up to 1 hour, and centrifuged at 4° C. for 5 min at 1000 rpm. Sera may be aliquotted and stored frozen at −80° C. Patient and control sera may be collected following a clinical protocol approved by Vanderbilt University Medical Center.

Chromatographic Separations. In selected cases, sera may be either fractionated by using reverse phase magnetic beads or the PPAS device described herein. Magnetic Bead Chromatography. Sera may be incubated with superparamagnetic, porous silica-based particles (<1 μm diameter; 80% iron oxide), surface-derivitized with C18. A suspension of C18/K magnetic particles (500,000 particles/μg; 50 μg/μL DD water) may be thoroughly mixed for 2 min. by vortexing to obtain homogeneous dispersion. Next, a 50 μL bead solution may be added to 50 μL of serum and mixed slowly by pipetting up and down five times. A magnet may then used to pull the beads to the side of the tube while the supernatant is removed via pipette and discarded. The beads may then be washed thoroughly with 200 μL of 0.1% TFA in water. Finally, the peptides may be step wise eluted from the particles with 5 μL volumes of 20% and then 70% acetonitrile by pipetting the beads up and down 10 times. 3 μL of the eluate may then be transferred to another tube, mixed with 6 μL of MALDI matrix solution, and 1 μL deposited for MS analysis. Monolithic Fractionation/Concentration of Sample Analytes by using a PPAS Device™. Protein and peptide analytes may be analysed by using the general protocols described iabove. Upon arrival, the serum aliquots may be immediately stored at −80 degrees ° C. For example, blood serum samples may be prepared by adding 250 μL of 16 mM ammonium bicarbonate and 250 mM L-histidine to 1 μg/μL octyl-b-D-glucopyranoside (OG), 0.5 μL glycerol and 10 μL of human serum in an Eppendorf microtube (500 μL volume). The resulting sample mixtures may be centrifuged at about 1000 g for 1 minute in order to bring together the mixture droplets. One-half of the samples may be adjusted to pH 7.0 and other half adjusted to pH 6.8. 160 mM ammonium bicarbonate and 250 mM L-histidine buffer may be used for the cartridge reservoir buffer (below the monoliths). All samples may be analyzed in five replicate runs.

For casting of capture materials 40 in capture slides 42, a carbon doped polypropylene (˜50,000 ohm/cm) slide containing a plurality of through holes is injection molded. The slide is then sandwiched between two soft silicon rubber gaskets, and two quartz plates. The functionalization solution described previously is placed via pipette into each of the through holes and illuminated using the Xenon Arc lamp fitted with a water filter for approximately 15 min. The substrate is then removed from the sandwich and the monolith solution, containing butyl methacrylate and 2-hydroxyethyl methacrylate, is added to each of the through holes as previously described, the sandwich reconstructed, and illuminated for 15 min. Following the casting procedure, the slide is soaked in 106 mM ammonium biocarbonate and 250 mM L-histidine for 30 minutes. Finally the monolith slide is thoroughly washed with deionized water. A cathode made of platinum may be placed directly into the sample wells. A platinum anode may be placed in contact with the buffer reservoir. The platinum electrodes may be connected to a custom-designed, multiplexed potentiostat and approximately 1 mA of current may be applied to the wells. The process may be allowed to proceed for about 20 minutes before the voltage is set to zero and the leads to the electrodes disconnected. One-half of the samples may be processed at +1 mA, and the other half at −1 mA, for approximately 20 minutes. During the course of each analysis, the current for each of the wells may be monitored and plotted. After the electro-concentration procedure is complete, the PPAS cartridge 2 may be disassembled and the cartridge capture slide 42 is washed to remove interfering species such as pH buffers and salts. Lastly, CHCA MALDI matrix may be applied and the slide directly analyzed via MALDI-MS.

For both the PPAS device protocol and the magnetic beads, 30 fmol (per peptide) and 500 fmol (per protein) of commercially available calibration standards (Bruker Daltonics) may also be mixed with CHCA matrix and applied separately onto the target plates, centrally located to six neighboring serum samples, together arrayed in a 3×2 pattern. Reproducibility may be determined to assess variability in: a) a single well in a single device, b) different wells of the same device, c) the same wells of different devices, and d) different wells of different devices. A factorial analysis may be used to determine any effects of well position, or interactions between the wells (or other variables).

Mass Spectrometry. Peptide profiles may be analyzed with Applied Biosystems Voyager DE and 4700 model MALDI mass spectrometers using the typical procedures: 20 kV accelerating voltage, 94.1% grid voltage, 0.050% guide wire voltage, 110 ns delay, 3000 laser setting, 64 scans averaged, 1.1 e-6 torr, 511 low mass gate, negative ions off. Spectra may be acquired in linear mode geometry.

Spectra acquired via the MS instrument's software may be processed using commercial Efeckta software (Efeckta Technologies, Corp., Steamboat Springs, Colo.). This software provides automated smoothing, baseline correction, and peak designation of spectra during acquisition. All data manipulations may made in accordance with techniques published by Tempst et al.^{xiv} After manually implemented external calibration, the peak (i.e., m/z) lists may be saved to a file in text file format required for subsequent statistical analysis (see below). Peak lists may be imported into the database for a series of data transformations. To first create a simple binary system for initial pattern analysis, peak intensities may be reduced to indicate the presence or absence in any of the resulting bins of the peptides observed in any particular sample.

Next, the peaks may be aligned across all samples within a particular set by binning within a window expanding proportionally with peptide mass (e.g., 1500 ppm). Binning is done by merging all m/z values from all samples into one long list, sorted by increasing value. The first mass is then marked as “real” and compared to the adjacent sorted masses. Any adjacent masses within a user-defined window are called “duplicate”. The process is repeated with the next larger m/z value that has yet to be marked until all the masses in the sorted list are tagged as either “real” or “duplicate”. “Duplicate” masses are then discarded. In the current application, the tolerance may be either 2 Da or 1500 ppm (0.15%), depending on the experiment. Note that the assignment of the first m/z value in each bin of masses as the “real” mass is arbitrary and is used solely as a designation for the bin. Once the m/z values are binned, a spreadsheet is automatically exported with the results. The first column will show a list of all the “real” masses surviving the binning process. The remaining columns will represent the samples and whether each sample has a peak binned with the corresponding “real” mass.

Statistical Data Analysis. After binning of m/z peaks across all samples of a study set, the Efeckta software may be used to evaluate proteomic data. A virtual “experiment” may be created in the software to represent the masses. The data may be normalized by using ubiquitin, and at least one other peptide peak found in all of the samples. In the parameter section of the experiment, the samples may be labeled as either Cancerous or Normal. In the Interpretation section, the Analysis mode may be set to “log of ratio” and all measurements used. Sample Names mayl be displayed as noncontinuous parameter. Once the experiment is created, the masses may be filtered using a one-way ANOVA nonparametric test (Mann—Whitney U test) and no multiple test correction at p<0.05. This test is meant to filter out masses that do not vary significantly across two different groups with multiple samples. The filter leaves behind masses that exhibit important changes between the prostate cancer and control groups. The changes may be confirmed by using two techniques: clustering and class prediction.

For the first technique, Efeckta's clustering tool may be used and its results displayed as a decision tree. On the x-axis, samples that are similar may be placed near each other on the tree. Similarity of samples will be assessed by Pearson correlation. Dissimilar samples will be placed apart from each other. On the y-axis, masses are grouped in the same way also using Pearson correlation to test for similarity. The clustering method discarded masses with no data for half the samples.

For the second confirmation, the filtered peptide masses from the nonparametric test will be also analyzed by class predictor algorithm, called k-nearest neighbor. To learn the accuracy of the class prediction, a cross-validation method, known as “leave-one-out”,^{6,19} will be implemented. It takes N−1 samples as a training set in the class predictor algorithm. The Nth sample is then used as a test set, and the process is repeated N times such that all samples are used as a test set once.

For MALDI MS analysis, the center cartridge assembly is affixed to a suitable MALDI mass spectrometry sample plate for introduction into a mass spectrometer. A small droplet of MALDI matrix dissolved in a suitable solvent is then added to the analyte capture regions of the capture membrane. The solvent is allowed to dissolve the analytes present at the capture sites on the capture membrane. As the solvent evaporates, the analytes become incorporated within MALDI matrix crystals that form on the top surface of the capture membrane. After allowing time for evaporation of the solvent liquid and formation of MALDI matrix crystals, the sample plate is ready for introduction into a MALDI mass spectrometer. Upon insertion of the MALDI sample plate into a mass spectrometer, the MALDI matrix crystals are illuminated with an intense UV laser light pulse resulting in ionization of a fraction of the analyte molecules. Ions from this fraction are measured based on their time of flight to the detector and plotted according to their mass-to-charge ratio and intensity.

Example Analysis of Proteins Present in Blood Serum:

FIGS. 3 and 4 show the results of utilizing a 25-well version of the PPAS, with single capture membrane, and subsequent analysis by MALDI mass spectrometry. Using the prototype PPAS, separations from an array of serum samples have been carried out simultaneously at relatively high speed (within 60 minutes). Subsequent reductions of the thickness of the separation layer from about 5 mm to about 1.0 mm, or less and increasing the voltage applied across the separation layer from about 1.0 to 10 volts to about 10 to 100 volts enables separation, concentration and capture in 10 minutes, or less. Electrophoretic concentration of selected fractions directly onto the disposable MALDI plate provides the additional benefit of increased MALDI-MS sensitivity and rapid differential expression profiling. A major problem with analyzing clinically important, low abundance peptides in blood, plasma, or serum is that high abundance proteins mask the appearance of low abundance peptides. Affinity removal of the most abundant proteins from blood, plasma or serum samples, however, has been hypothesized to also remove a significant number of low abundance, hydrophobic peptides. In these studies, serum samples were treated with MALDI-compatible detergents in order to promote dissociation and subsequent separation and concentration using the PPAS and detection via MALDI-MS. Examples of such MALDI-compatible detergents are those of neutral charge, such as Triton X-100, octyl glucoside, NP-40, or the like. Such neutral detergents do not electrophoretically concentrate in the PP capture layers.

The results from MS analysis of PP mixtures may be compared to purified PP standards (e.g., a sample containing only ubiquitin, cytochrome C, insulin and 1% TFA). The standard samples may be diluted directly into 0.1% TFA (to either 800 femtomol/μL or 10 femtomol/μL) so that little, or no, interfering species are present after evaporation of the solvent prior to analysis by MALDI-MS. Alternatively, PP labeled with chromophoric or fluorophoric labels may be incorporated as standards. For example fluorophoric moleucules may be labeled with Fluorescein (F) Texas Red (TR), Rhodamine (Rh) or Marina Blue (MB) by employing reagents and methods well known to those skilled in the art of protein modification. Thus either 0.2 μL of TR-ubiquitin, MB-bovine serum albumin (MB-BSA), each at 1-2 μg/μL, may be incorporated into a 2 μL sample containing 250 mM aqueous L-histidine buffer with 25% (w/v) glycerol.

The results shown in FIGS. 3 and 4 were obtained with a polyacrylamide layer used to remove high molecular weight, high abundance proteins from human serum. No albumin was observed (at m/z of 68,000) in the spectra shown in FIGS. 3 and 4. These results show that the polyacryamide layer effectively removes serum albumin, as MALDI-suppressing interference, from the mixture. When the electrophoresis run time was extended to over 1 hour, however, the beginning of an albumin signal was observed. Concomitantly, a reduction in intensity of the other captured proteins is observed presumably due to the well-know suppression of ionization of lower abundance proteins in the presence of the high abundance albumin. For analysis of high molecular weight proteins in the PPAS the polyacrylamide layer may be replaced by a non-sieving agarose layer (and high abundance proteins removed by alternative treatments, e.g. by affinity chromatography).

The PPAS invention captures proteins and polypeptides onto a solid-phase capture membrane, allowing salts and other interfering molecules to be washed away. Then upon application of a MALDI matrix solution to the membrane, the proteins are released and are incorporated into MALDI matrix crystals that precipitate on the membrane surface. After MALDI matrix addition, the membrane is dried and inserted directly into a MALDI-MS instrument for quantification of mass and relative abundance of the attached proteins.

The PPAS may utilize just one capture membrane with (only limited) fractionation into either positively charged or negatively-charged molecules at the selected separation pH. The PPAS with one capture membrane provides for removal of high-abundance proteins (either by an incorporated sieving layer, or carried out in a preliminary step). No other fractionation need be performed. Optionally two, or more capture membranes may be employed in series to further increase the fractionation. Because MALDI-MS is subject to suppression of sample ionization by high abundance molecules, such an increase in fractionation increases the sensitivity approximately in proportion to the fractionation performed.

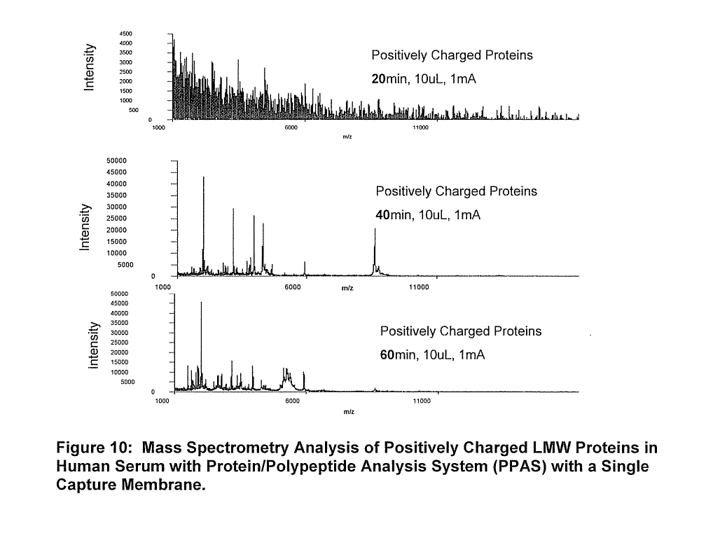

A basic example of the invention is shown with an alpha prototype system. The prototype system has a 5×5 array of 25 of capture wells and allows 25 samples to be electrophoretically separated and captured simultaneously in a signle cartridge. For the mass spectrometery results shown in FIG. 10, the sample pH was 7.8 and the current in each well was set to 1 ma for the times indicated. After capture of the proteins, the membranes were washed in DI water and then released by application of a MALDI matrix solution comprised of one volume of 0.1% trifluoroacetic acid solution saturated with alphacyano-4-hydroxycinnamic acid (CHCA) and one volume of acetonitrile. The matrix was then allowed to dry and placed in a MALDI mass spectrometer for analysis. The time course shows that between 20 and 40 minutes was required for arrival of the initial positively charged proteins and that additional proteins arrived between 40 and 60 minutes. Not shown are data that indicate that there is no substantial change in the captured proteins observed subsequent to 60 minutes. All MALDI-MS analyses were performed with an ABI/Perceptive Biosystems Voyager DE (MALDI-TOF) instrument by using an in-house designed QGEN_PR2 method. For use with CHCA matrix solutions typical spectrometer settings were: 20 kV accelerating voltage, 94.1% grid voltage, 0.050% guide wire voltage, 110 ns delay, 3000 laser setting, 64 scans averaged, 1.1e-6 torr, 511 low mass gate, negative ions off. For use with the sinapinic acid matrix solutions, typical spectrometer settings were: 25 kV accelerating voltage, 92.0% grid voltage, 0.30% guide wire voltage, 200 ns delay, 3800 laser setting, 64 scans averaged, 1.67e-6 torr, 1000 low mass gate, negative ions off.

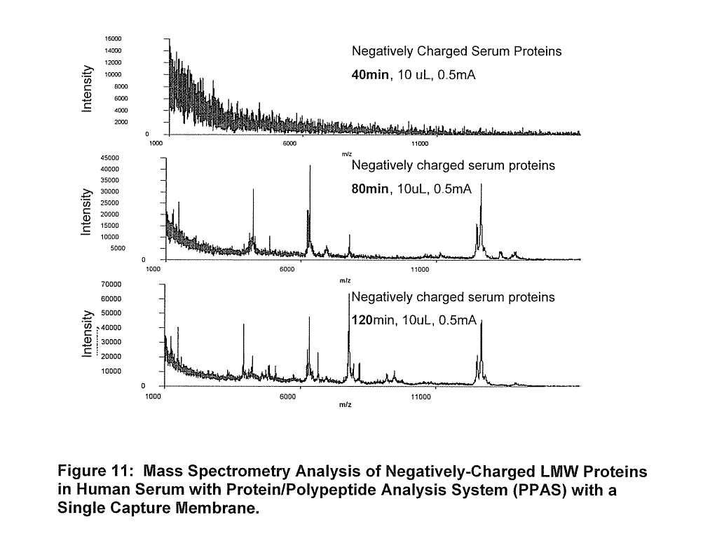

For the MALDI mass spectrometry results shown in FIG. 11, the procedure and analysis were similar to those described in FIG. 10, except that the polarity of the electrodes was reversed. Thus the proteins observed under the two conditions (of reversed polarity) clearly are different, in accordance with the fact that the native charge of the proteins observed in the two spectra are opposite at the predetermined pH of the sample (i.e., 7.8 in this case). Similar to the results with the positively-charged proteins (FIG. 10), the time course for capture of the negatively charged proteins shows that between 40 and 80 minutes was required for arrival of the initial negatively-charged proteins and that additional proteins arrive between 80 and 120 minutes. Also not shown are data that indicate that there is no substantial change in the captured proteins observed subsequent to 120 minutes. (Note that the current levels for the experiment shown in FIG. 10 are twice as large as the current levels employed in the experiment shown in FIG. 11. Conversely the electrophoresis times shown in FIG. 10 are half of those shown in FIG. 11, i.e. the number of coulombs of charge transfer employed during electrophoresis (for twice the period of time) are identical to those at half the time, shown in FIG. 10. (Thus the charge transferred in the two experiments shown

Further methods for Utilizing the Subjject Invention for MALDI-Mass Spectrometery Analysis

MALDI matrix may be prepared by using previous published methods subsequeintly may be applied to the cartridge capture slides 42 by using one of the following general methods: 1) manual pipette application, 2) application using a commercial liquid handling workstation, 3) spray coating, 4) cartridge capture slide immersion in matrix solution. Concentrated matrix solution is applied is order to achieve a matrix-to-analyte ratio acceptable for MALDI analysis.

One particularly useful application comprises depositing on the monolith surface a solution of Sinapinic acid (20 mg/mL in 50:50 acetonitrile/0.1% trifluoroacetic acid). A volume of 0.25 uL of this solution is applied to the top of the cartridge capture slide using a micropipette. This solution is dried at room conditions over the course of approximately 5 minutes, at which time an additional 0.25 uL of matrix is applied. The slide is allowed to dry at room conditions or in a vacuum desiccator.

Customarily, after MALDI matrix deposition and drying, capture slides are introduced into mass spectrometers according to instrument manufacturers specifications. The slides are designed to fit into a specially designed sled that adapts the cartridge capture slide to the x-y sample stage of the MALDI mass spectrometer. The sled is designed to conform to the following requirements: A) The cartridge capture slide must be held perpendicular to the axis of ion extraction inside the mass spectrometer, B) the sled must interface with the cartridge capture slide in a way that provided a path for the dissipation of surface charging of the cartridge capture slide, C) the cartridge capture slide surface height must match that of each instruments standard sample carrier, and D) the position of each monolith relative to the sled must always be the same. Each of these requirements is known to one skilled in the art of mass spectrometry.

Mass spectra of analytes captured on the capture slides 42 are processed in a standard fashion by using sets of tools available commercially and well known to those skilled in the art of such analysis. For example, baseline subtraction, normalization, peak detection, and spectral alignment are performed by using software commercially available as ProTS-Data (Efeckta Technologies, Inc.; Steamboat Springs, Colo.; Version 1.1.1.0) The data analysis, in summary is as follows:
1. Background estimation/subtraction: Background signal is estimated by using robust, local, statistical estimators. As background is essentially “noise” and does not contain biologically relevant information and varies from spectrum to spectrum, amplitude information needs to be made more comparable by subtracting the value of the background from each spectrum.
2. Normalization: The amount of sample ionized can fluctuate from spectrum to spectrum, due to changes in laser power, variations in the amount of ionizable sample, and variations in the positioning of the laser on the MALDI plate. To obtain more reliable quantitive information on the peak amplitudes spectra are normalized to the total ion current.
3. Peak picking: The noise estimators are calculated and used to identify peaks in a spectrum and to assign a reliable estimate of their signal/noise ratio. For a typical MALDI spectrum from tissue samples we typically detect between 100 and 200 peaks with a signal/noise ratio cutoff of 3.
4. Spectral alignment: The absolute mass scale of single spectra can vary considerably. A selection of common peaks can then be used to register spectra to a common m/z scale.
   Classifier Generation and Validation

Within standard mass spectroscopy analyses, mass peak lists (containing the centroid values and normalized intensities) are constructed and then exported to individual data files. A variety of Software tool sets facilitate the detection of biomarkers from mass spectra from these data. The Software at the same time provides rigorous tools for the assessment of statistical significance across different populations with a common variance. While feature ranking gives some idea about the importance of features for discriminating groups, a more thorough analysis requires the use of features in a supervised learning procedure. In supervised learning one provides a category label for each instance in a training set, i.e., each spectrum, and seeks to reduce the number of misclassifications. A large variety of procedures have been developed to address supervised learning problems. The output of supervised classification algorithms generally may be used as a classifier (dependent on the training set) that generates a class label for a new instance or spectrum. See for example A. Webb, A. John Wiley & Sons Ltd., 2002, Statistical pattern recognition; and also B. Duda, O. R., Hart, P. E., Stork, D. G., Wiley & Sons Ltd., 2001, Pattern Classification.

The invention has been described with reference to various specific and preferred embodiments and techniques. However, it should be understood that many variations and modifications may be made while remaining within the spirit and scope of the invention.

We claim:

1. A device for electrophoretically separating, concentrating, and capturing an analyte in a sample comprising:
a sample well for retaining a fluid sample in an electrolyte;
a separation layer providing a path for diffusive ionic, and fluidic communication with the well; and
a capture layer providing a path for diffusive ionic, and fluidic communication with the separation layer.

2. The device of claim 1 wherein the sample well comprises a top opening, side walls, and a bottom opening, and wherein the sample well is progressively reduced in dimension from the top opening to the bottom opening.

3. The device of claim 1 wherein the sample well has a total interior volume of up to about 400 μL.

4. The device of claim 1 comprising a plurality of sample wells disposed as an array of sample wells.

5. The device of claim 1 wherein the separation layer is comprised of a gel.

6. The device of claim 5 wherein the gel is a polyacrylamide gel.

7. The device of claim 1 wherein the separation layer is disposed within an aperture disposed in a cartridge gel plate.

8. The device of claim 1 wherein an array of substantially identical separation layers are disposed within an array of substantially identical apertures disposed in a cartridge gel plate.

9. The device of claim 8 wherein the cartridge gel plate is manufactured of an electrically insulating thermoplastic polymer.

10. The device of claim 8 wherein the separation layers are covalently bound to the cartridge gel plate at the apertures of the cartridge gel plate.

11. The device of claim 1 wherein the capture layer is a porous material.

12. The device of claim 1 wherein the capture layer is a hydrophobic porous polymer, a hydrophilic porous polymer, or a mixture of hydrophilic and hydrophobic polymers.

13. The device of claim 1 wherein the capture layer is a porous poly(vinylidene difluoride) material.

14. The device of claim 1 wherein the capture layer is disposed within an aperture disposed in a cartridge capture slide.

15. The device of claim 1 wherein an array of substantially identical capture layers are disposed within an array of substantially identical apertures disposed in a cartridge capture slide.

16. The device of claim 15 wherein the cartridge capture slide has low electrical conductivity so as to provide for dissipation of static charge.

17. The device of claim 15 wherein the cartridge capture slide has a volume resistivity of between about 10⁴ and about 10⁸ ohm-cm.

18. The device of claim 15 wherein the cartridge capture slide has a flatness, or is capable of being flattened when inserted into a MALDI mass spectrometer, to +/−50 microns.

19. The device of claim 15 wherein the cartridge capture slide is about 5.3 mm long, about 3.5 mm wide, and about 1 mm thick.

20. The device of claim 15 wherein the substantially identical apertures in the capture slide are substantially circular and are about 0.5-1 mm in diameter.

21. The device of claim 15 wherein two or more cartridge capture slides are stacked in series such that the capture layers in one cartridge capture slide are aligned with corresponding capture layers in an adjacent cartridge capture slide.

22. The device of claim 21 wherein the capture layers in one cartridge capture slide are in fluid and electrical communication with corresponding capture layers in an adjacent capture slide.

23. A device for capturing a sample analyte for analysis in a mass spectrometer comprising:
a cartridge capture slide comprising a plurality of apertures disposed therein;
a plurality of capture layers disposed in the plurality of apertures.

24. The device of claim 23 wherein the capture layers manufactured from a porous material.

25. The device of claim 24 wherein the porous material is a hydrophobic porous polymer, a hydrophilic porous polymer, or a mixture of hydrophilic and hydrophobic polymers.

26. The device of claim 23 wherein the capture layers are porous poly(vinylidene difluoride).

27. The device of claim 23 wherein the plurality of apertures containing the plurality of capture layers are arranged as an array.

28. The device of claim 27 wherein the array comprises 96 apertures.

29. The device of claim 23 wherein the cartridge capture slide has low electrical conductivity so as to provide for dissipation of static charge.

30. The device of claim 23 wherein the cartridge capture slide has a volume resistivity of between about 10⁴ and about 10⁸ ohm-cm.

31. The device of claim 23 wherein the cartridge capture slide has a flatness, or is capable of being flattened when inserted into a MALDI mass spectrometer, to +/−50 microns.

32. The device of claim 23 wherein the cartridge capture slide is about 5.3 mm long, about 3.5 mm wide, and about 1 mm thick.

33. The device of claim 23 wherein the cartridge capture slide wherein the plurality of apertures in the capture slide are substantially circular and are about 0.5-1 mm in diameter.

34. A method for identifying an analyte by mass spectrometric analysis comprising:
providing the device of claim 1;
placing a sample fluid containing an analyte in the sample well;
applying an electrical current to the sample fluid to effect electrical transport of the analyte through the separation layer and onto the capture layer; and
identifying the mass of analytes on the capture layer in a mass spectrometer.

35. A cartridge capture slide adaptable for use in a mass spectrometer comprising:
an array of apertures disposed in the cartridge capture slide; and
an array of capture layers disposed in the array of apertures,
wherein the cartridge capture slide is incorporated within a device comprising an array of sample wells and a cartridge gel plate, and wherein the cartridge gel plate comprises an array of apertures in which are disposed an array of separation layers.

36. The cartridge capture slide according to claim 35 having a volume resistivity of between about 10⁴ and about 10⁸ ohm-cm.

37. The cartridge capture slide according to claim 35 having a flatness, or capable of being flattened when inserted into a MALDI mass spectrometer, of +/−50 microns.

38. The cartridge capture slide according to claim 35 having length of about 5.3 mm, width of about 3.5 mm, and thickness of about 1 mm.

39. The cartridge capture slide according to claim 35 wherein the plurality of apertures are substantially circular and are about 0.5-1 mm in diameter.
